(19) United States
(12) Patent Application Publication
(10) Pub. No.: US 2018/0320242 A1
(43) Pub. Date: Nov. 8, 2018

(54) MICROBE ASSOCIATIONS THAT HAVE NEW OR IMPROVED CHARACTERISTICS

(71) Applicant: UNIVERSITY OF COPENHAGEN, Copenhagen (DK)

(72) Inventors: Mette Nicolaisen, Frederiksberg C (DK); Stefan Olsson, Frederiksberg (DK); Ole Nybro, Nivoe (DK); Behnoushsadat Ghodsalavi, Birkeroed (DK); Deborah Jean Springer, Durham, NC (US); Michael A. Furlan, Durham, NC (US)

(73) Assignee: UNIVERSITY OF COPENHAGEN, Copenhagen (DK)

(21) Appl. No.: 15/772,571

(22) PCT Filed: Nov. 7, 2016
(86) PCT No.: PCT/EP2016/076804

§ 371 Date: May 1, 2018

(30) Foreign Application Priority Data
Nov. 6, 2015 (EP) 15193427.0

(51) Int. Cl. C12R 1/80; A01N 63/04; A01H 3/00; C12R 1/07

(52) U.S. Cl. CPC C12R 1/80; A01N 63/04; C12N 1/14; C12R 1/07; A01H 3/00

(57) ABSTRACT

Disclosed are methods for identifying microbes that associate with other microbes, and possess new or improved characteristics. Also disclosed are microbes isolated using the methods. Also disclosed are methods for using the microbes.

### REFERENCE TO A DEPOSIT OF BIOLOGICAL MATERIAL

This application contains a reference to a deposit of biological material, which deposit is incorporated herein by reference. For complete information see last paragraph of the description.

### BACKGROUND

Although a variety of different microbiomes have been identified, and microbial diversity within many of these microbiomes has been characterized, interactions between different microbes in these microbiomes, called microbial consortia, are not well understood. For example, even though one gram of soil may contain millions to billions of microbes, and roots of a plant growing in soil may harbor tens of thousands of different microbial species, little is known about the effects produced by interactions between specific microbes in these microbiomes.

### SUMMARY

Disclosed herein are methods for identifying microbes that associate with one other and result in new and/or improved properties, functions and/or characteristics, as compared to the microbes when not associated with one another. We have found that a first microbe, that associates with a second microbe, and affects growth or proliferation of the second microbe, and/or whose growth or proliferation is affected by association with the second microbe, can produce characteristics that are not found in either of the two microbes alone (e.g., when the two microbes are not associated with each another). In one example, the microbes identified are from environmental samples. In one example, the methods exemplify natural systems (e.g., soil microcosm) and the microbial consortia within natural systems.

In one example, methods are disclosed for isolating bacteria that associate with a fungus, and for screening the bacteria for the capability to affect growth of the fungus and/or for the ability of the fungus to affect growth of the bacteria. The methods for isolating the bacteria may mimic close-to-natural systems. In one example, the screening methods may identify bacteria that can stimulate fungal growth (and/or the fungus may stimulate bacterial growth). In one example, the screening methods may identify bacteria that impede fungal growth (and/or the fungus may impede bacterial growth). Bacterial/fungal associations may be tested for new and/or improved characteristics. In one example, the fungus may include a non-mycorrhizal fungus. In one example, the fungus may include a fungus capable of solubilizing phosphate. In one example, the fungus may include Penicillium. In one example, the fungus may include Penicillium bilaiae. In one example, the Penicillium bilaiae may include strain P-201. In one example, the Penicillium bilaiae may include both strains P-201 and P-208.

In one example, bacteria that associate with a fungus are isolated by establishing the fungus on a support (e.g., a glass support), contacting the support with a sample from an environment (e.g., a soil microcosm), and obtaining bacteria that associate with the fungus. The method generally may be performed under conditions that simulate a natural system (e.g., under close-to-natural conditions). The support may be washed to remove bacteria that are not associated with the fungus. Bacteria attached to the fungus may be cultured.

Bacteria associated with the fungus may be screened for their capability to affect fungal growth (and/or may be screened for the ability of the fungus to affect bacterial growth). In one example of the screening, the fungus and a bacterium may be placed on a support (e.g., agar plates), proximate to one another, but not contacting one another, so an effect of the bacterium on growth of the fungus (and/or fungus on bacterium) is capable of being detected. In one example, the screening is performed under conditions where nutrients in addition to those provided by a concentration of agar are not provided to the bacteria or the fungus (e.g., water-agar plates may be used).

Bacteria that have been shown to affect growth of the fungus (and/or bacteria whose growth is affected by the fungus), may be associated with the fungus, and the association of bacterium and fungus may be tested for characteristics other than an effect of the bacteria on growth of the fungus and/or an effect of the fungus on growth of the bacteria (e.g., secondary characteristics; primary characteristic is effect on growth). The testing may reveal associations of bacteria and fungus that possess new or improved properties, functions and/or characteristics that are not present when the bacteria and fungus are not associated. In various examples, the bacteria may cause the fungus to produce the characteristic(s), the fungus may cause the bacteria to produce the characteristic(s), or the bacteria and fungus may both contribute to the characteristic(s). Other mechanisms by which the characteristics are produced may exist.

In one example, association of a bacterium with a fungus, where the fungus is capable of solubilizing phosphate alone, may result in the fungus increasing the amount of phosphate it can solubilize and/or increasing the rate at which the fungus can solubilize phosphate, as compared to phosphate solubilization by either the bacterium or fungus alone. Other secondary characteristics, besides phosphate solubilization, or in addition to phosphate solubilization, may be identified. In one example, a bacterium associated with a fungus may increase capability of the bacterium and fungus to facilitate plant growth, as compared to facilitation of plant growth by either the bacterium or the fungus alone. Such bacteria, isolated using the methods described herein, may be called “helper bacteria.” These bacteria may help or facilitate properties of the fungus.

Also disclosed are compositions of bacterial strains isolated using the disclosed methods. In one example, the bacterial strains may include one or more isolated bacterial strains 313 (DSM 32170), 346 (DSM 32171), 351 (DSM 32172), 365 (DSM 32173) and 371 (DSM 32174). The compositions containing the strains may be solid or liquid compositions. The compositions may include concentrations of the bacterial strains that are higher than concentrations of the bacterial strains found in nature. The compositions may contain concentrations of bacterial spores from the bacterial strains that are higher than concentrations of the bacterial spores found in nature (and/or higher ratio of spores to vegetative cells than found in nature). In one example, the compositions may include one or more of the 313, 346, 351, 365 and 371 bacterial strains, and one or more strains of Penicillium bilaiae. The Penicillium bilaiae contained in the compositions may contain concentrations of vegetative cells higher than concentrations of vegetative cells found in nature. The Penicillium bilaiae contained in the compositions may contain concentrations of spores higher than found in nature (and/or higher ratio of spores to vegetative cells). In one example, the compositions may also include one or more excipients.

Also disclosed are methods for using the compositions of the bacteria, or for using the compositions of the bacteria and the fungus (e.g., Penicillium bilaiae). In one example, the compositions may be supplied to a plant. Supplying to a plant may include applying the composition to a seed, which may be planted and grown, or applying the composition to a furrow in which a seed or seedling is planted and grown. In one example, the compositions supplied to a plant may also include biostimulants, nutrients, pesticides or plant signal molecules. The compositions, when supplied to plants, may facilitate growth of the plants.

### BRIEF DESCRIPTION OF THE DRAWINGS

In the accompanying figures, which are incorporated in and constitute a part of the specification, methods related to identifying microbes that associate with each other and produce improved or new characteristics, microbes isolated using the methods, and methods for using the microbes are disclosed. Changes, modifications and deviations from the disclosures illustrated in the figures may be made without departing from the spirit and scope of what is claimed, as disclosed below.

FIG. 1 illustrates a schematic diagram of example steps in a close-to-natural system for isolating bacteria that form associations with a non-mycorrhizal fungus. See Example 1 for details.

FIG. 2 illustrates two example light micrographs (A and B) of SYBR® Green staining of cover slides after the process described in Example 1 and illustrated in FIG. 1. Both Penicillium hyphae (elongate structures) and bacteria attached to the hyphae (relatively more intense-staining particles localized to hyphae exterior) are visible.

FIG. 3 illustrates example colony counts from cover slides that contained hyphae (A) and control cover slides that did not contain hyphae (B), from the process described in Example 1.

FIG. 4 illustrates example UP-PCR banding patterns (A and B) from randomly selected bacterial isolates obtained from the process described in Example 1.

FIG. 5 illustrates examples of the assay used to score bacterial effects on Penicillium growth. Panel A is a schematic drawing of the assay, showing the fungal plug in the center of the circle, the circle representing a culture plate (e.g., petri dish) and the line of bacteria streaked on one side of the plate, a short distance from the fungal plug. FIGS. 5B-D are pictures of example implementations of the method. The circles designate the fungal plugs. The arrows (and length of the arrows) indicate effects of the bacteria on growth of the fungus from the plug. FIG. 5B shows an example of a bacterium that had a negative effect on Penicillium growth. FIG. 5C shows an example of a bacterium that had a neutral effect on Penicillium growth. FIG. 5D shows an example of a bacterium that had a positive effect on Penicillium growth.

FIG. 6 illustrates example micrographs of interactions of bacterium and fungus on water agar plates that showed a positive effect of the bacteria on fungal radial growth, using the assay described in Example 2. Panel A shows low magnification. Panel B shows high magnification.

FIG. 7, panel A shows a plate of Penicillium that was incubated for 8 days with one of the five bacterial strains selected based on a positive effect on fungal radial growth. The boxed area in panel A is shown at day 15, after SYBR® Green straining, at high magnification in a fluorescence micrograph in panel B. In panel B, bacteria are shown as the smaller, circular particles on the surface of the hyphae.

FIG. 8, panel A shows an example split water agar plate, with the 365 bacterial strain streaked on the right side of the plate, and Penicillium bilaiae mycelium on the left side of the plate. Panel B shows an example control split water agar plate with no bacteria on the right, and Penicillium bilaiae mycelium on the left.

FIG. 9 shows data from example fungal spore germination experiments. Panel A shows germinated Penicillium bilaiae spores, under conditions where the spores were not incubated with bacteria. Panel B shows germinated spores, under conditions where the spores were incubated with bacteria that facilitate growth of the fungus.

FIG. 10 illustrates example plates from organic phosphate solubilization experiments on calcium phytate agar plates. Panel A shows fungal mycelium without bacteria added. Panel A shows fungal mycelium that had bacteria added. Both panel A and B show the zones of phosphate clearing around the colonies.

FIG. 11 illustrates example plates from inorganic phosphate solubilization experiments on Sperber agar plates. Panel C shows fungal mycelium without bacteria added. Panels A and B show fungal mycelium that had added bacteria. All panels show the zones of phosphate clearing around the colonies.

### DETAILED DESCRIPTION

### Definitions

The following includes definitions of selected terms and phrases that may be used in the disclosure and in the claims. Both singular and plural forms of the terms and phrases fall within the definitions.

As used herein, “affect” or “affecting” means to have an effect on, or to change something.

As used herein, “alone,” in reference to a microbe, means that the microbe is not in an association with another microbe.

As used herein, “anti-fungal,” generally with reference to an agent (e.g., chemical or compound), means destructive to a fungus (e.g., fungicidal), or impeding growth or proliferation of a fungus (e.g., fungistatic).

As used herein, “anti-germinant,” generally with reference to the effect a substance may have on bacterial and/or fungal spores, means the substance inhibits or partially inhibits a spore from germinating or entering a vegetative state.

As used herein, “applying,” generally with reference to a composition, means to place the composition on, in or in close proximity to something.

As used herein, “associates with,” with reference to a microbe (e.g, bacterium) that associates with another microbe (e.g., fungus), means that the bacterium combines with the fungus.

As used herein, “association,” with reference to an association of a microbe (e.g., bacterium) and another microbe (e.g., fungus), means the bacterium and fungus are together (e.g., combined).

As used herein, “attached to,” with reference to a microbe (e.g., bacterium) that is attached to another microbe (e.g., fungus), means that the bacterium fastens or affixes to the fungus.

As used herein, “binds to,” with reference to a microbe (e.g., bacterium) that binds to another microbe (e.g., fungus), means that the bacterium coheres or secures to the fungus.

As used herein, “capable of” or “capability to,” refers to the ability or capacity to do or achieve a specific thing.

As used herein, “characteristics,” refers to one or more, or a combination of, properties, traits, or functions of an organism.

As used herein, “close-to-natural system,” generally means a laboratory-based system for identifying and/or isolating microbes that is designed to exemplify or mimic a microbiome found in nature, and the microbial consortia within the natural microbiome. One example close-to-natural system is designed to mimic a soil-based microbiome. “Close-to-natural conditions” means that individual parameters of the laboratory-based system are the same as/near to the conditions in the corresponding natural system.

As used herein, “colony forming units” or “CFU,” refers to individual colonies of microorganisms. Generally, CFU are units used to estimate the number of viable microbes in a sample. In one example, microbes are applied to a solid or semi-solid growth medium (e.g., containing agar) at a density at which single microbes that proliferate and form visible colonies can be counted. The visible colonies are called colony forming units or CFU.

As used herein, “colonize,” with reference to a microbe (e.g., bacterium) that colonizes another microbe (e.g., fungus), means that the bacterium is present or established on (or in) the fungus. In one example, colonize means that the bacterium may proliferate on (or in) the fungus.

As used herein, “combination,” with reference to a combination of a microbe (e.g., bacterium) and another microbe (e.g., fungus), means that the bacterium and fungus are in proximity to one another or used together. “Combining” refers to an action in placing the bacterium and fungus in proximity to one another and/or an action in preparation for using the bacterium and fungus together.

As used herein, “contact,” with reference to two or more objects, means that the objects physically touch each other. “Contacting” refers to an action whereby two or more objects are made to touch each other.

As used herein, “container,” means an object that can be used to hold, transport, store or house something.

As used herein, “culturing,” with reference to a microbe, means an action to grow or propagate a microbe.

As used herein, “detect,” means observe or discover.

As used herein, “enhance,” means to improve or make better. In one example, a bacterium may enhance or improve the ability of a fungus to solubilize phosphate.

As used herein, “enriching,” with reference to a microbe (e.g., bacterium), means an action to increase the proportion of a bacterium or bacteria. Generally, the enriching is directed to increasing the proportion of bacteria that have a specific property or can perform a specific function.

As used herein, “environmental samples,” generally means a sample from an environment, or a part of an environment. In one example, an environmental sample from a soil environment may be a handful or cupful of soil.

As used herein, “excipient,” means a substance that is included in a composition (e.g., a composition of a microbe or microbes), generally to aid, protect, support or enhance other components of the composition (e.g., the microbes). Example excipients may include, but are not limited to, carriers, polymers, wetting agents, drying agents, surfactants, anti-freezing agents, and the like. Excipients generally may be naturally occurring or non-naturally occurring. One type of non-natural excipient may be a synthetic excipient.

As used herein, “establishing,” means an action to settle into a position, or make secure in a certain place.

As used herein, “examining,” means an action to test, inspect or investigate.

As used herein, “express,” with regard to a characteristic of an organism, means that the characteristic is visible, observable or measurable.

As used herein, “facilitate” or “facilitation” of, for example, microbe or plant growth, refers to something that generally improves growth, as measured by one or more factors or properties, as compared to a standard or control. In one example, growth may be improved about 10%, 20%, 30%, 40%, 50%, 60%, 70%, 80%, 90%, 100% or more, compared to the standard or control.

As used herein, “furrow,” means a groove or trough in the ground, in one example, made by a plow.

As used herein, “gel,” means a jellylike substance.

As used herein, “germination,” means the process by which a spore (e.g., bacterial spore, fungal spore) enters the vegetative state (e.g., where the cells can divide).

As used herein, “granule,” means a small particle of a substance.

As used herein, “growth,” with respect to a microbe (e.g., bacterium, fungus) and/or plant, refers to an increase in size and/or number, development and/or maturation. “Growing,” in reference to a seed or seeding, refers to an action to cause growth.

As used herein, “hyphae,” means a branching, filamentous structure of a fungus in a vegetative state.

As used herein, “impeding,” means an action to retard or hinder.

As used herein, “inorganic phosphate,” refers to a phosphate that does not contain carbon.

As used herein, “insoluble phosphate,” refers to salts containing phosphorus that are not water soluble or minimally water soluble.

As used herein, “isolated,” means separated from or solitary. “Isolating,” means an action to obtain something that is isolated.

As used herein, “kit,” refers to a set or collection of two or more things, generally for use in a purpose. The two or more things that are part of a kit may be said to be “packaged” into or as a kit.

As used herein, “liquid,” refers to a state of matter that flows freely, has a definite volume and no fixed shape (e.g., it takes the shape of a container in which it is housed). An example liquid is water.

As used herein, “marketed,” refers to all or part of a process whereby something is sold or exchanged. For example, a marketed product may be advertised, promoted, distributed, offered for sale, sold, and the like. “Marketing,” refers to an action to market a thing.

As used herein, “medium,” with reference to a growth or culture medium for a microbe, refers to compositions for supporting growth. Example growth medium may include broths or agar plates.

As used herein, “microcosm,” means a small environment (e.g., in the laboratory) that is representative of a larger environment. In one example, a microcosm may be a soil microcosm that contains soil that is representative of soil in a field or plot.

As used herein, “microorganism” or “microbe,” means microscopic organisms, generally too small to be viewed by the naked eye. Example microorganisms include bacteria, archaea, protozoa, and some fungi and algae.

As used herein, “mixture,” means a combination of different elements, substances, microbes, and the like.

As used herein, “mycorrhiza,” refers to certain symbiotic associations of a fungus and roots of vascular plants. A fungus referred to as a “mycorrhizal” fungus is able to form such an association with plant roots.

As used herein, “non-mycorrhizal,” with reference to certain fungi, means fungi that are not capable of forming symbiotic associations with plant roots that are characteristic of mycorrhizal fungi.

As used herein, “nutrients,” with reference to nutrients for microbes (e.g., bacteria, fungi) or plants, refers to substances needed or useful for growth and/or maintenance of life. Herein, “additional nutrients” refers to nutrients in addition to, or other than, nutrients present in bacteriological-grade agar, used in preparing microbiological culture media, generally at about a concentration of about 0.5-1.5% of agar (weight/volume) of media.

As used herein, “obtained,” means to get, acquire or secure something. “Obtaining,” refers to an action to get, acquire or secure something.

As used herein, “offering for sale,” generally refers to an action where one party presents an option to a second party to acquire a product or service, and where the second party is free to accept or reject the product or service presented.

As used herein, “organic phosphate,” refers to a phosphate that contains carbon.

As used herein, “pathogen,” with reference to a plant pathogen, refers to an infectious and/or biological agent that is capable of causing disease, impeding growth or killing a plant.

As used herein, “phosphate,” generally refers to a salt or ester of phosphoric acid or related anion.

As used herein, “placing,” with reference to placing a microbe (e.g., fungus and/or bacterium) (e.g., on a support), refers to an action to put a thing in a place or location.

As used herein, “plant,” means a living organism that typically grows in soil, absorbing water and inorganic substances through roots and synthesizing nutrients by photosynthesis. Plant includes all plants and plant populations, such as desired and undesired wild plants or crop plants (including naturally occurring crop plants). Typical plants may include trees, shrubs, herbs, grasses, ferns, mosses, flowers, fruit, vegetables, houseplants and others. A plant may include the entirety of a plant or may include one or more forms, parts and/or organs of a plant, above or below ground.

Plant includes all plant forms, parts and/or organs which may include, for example, shoots, leaves, flowers, roots, needles, stalks, stems, flowers, fruit bodies, fruits, seeds, roots, tubers, rhizomes, and the like. Plants may also include harvested material and vegetative and generative propagation material (e.g., cuttings, tubers, rhizomes, off-shoots and seeds, etc.).

Use of the word “plant” as a verb (e.g., “planting”), with reference to a planted seed or seedling, or planting a seed or seedling, refers to placing or locating a seed or seedling in an environment (e.g., soil) where the seed or seedling can grow.

As used herein, the term “plant growth” means all or part of the process that begins with a plant seed and continues to a mature plant. Generally, as a plant grows and/or matures from a seed planted in soil, the seed germinates, the plant emerges from the soil, and roots, stems and leaves form. Generally, as a plant grows, it will increase in size and mass (e.g., yield). Plant growth may be determined by observing one or more aspects of a plant. For example, growth rate, amount of yield, root number, root length, root mass, root yield, leaf area, plant stand, plant vigor, number of pods, pod weight, plant weight, or any of a number of other factors, individually or collectively, may be properties that may be observed and may correlate with plant growth.

As used herein, “phosphate solubilization” or “solubilization of phosphate” generally refers to conversion of water-insoluble phosphates to water-soluble phosphates.

As used herein, “powder,” means fine, loose particles.

As used herein, “primary characteristic,” generally with reference to one or more characteristics of a microbe, means growth of the microbe.

As used herein, “promote,” with reference to the ability of a microbe (e.g., bacterium) to promote growth of another microbe (e.g., fungus), refers to the bacterium being able to positively affect growth of the fungus (e.g., cause the fungus to grow faster, easier, to a higher density, in the presence of less nutrients, and the like).

As used herein, “property,” with reference to a property of a microbe (e.g., fungus), refers to an attribute, quality, characteristic, function, and the like, of the fungus. Example properties of fungi include, but are not to be limited to, growth, and to solubilization of phosphate.

As used herein, “provide,” means to furnish, supply, allocate or distribute a thing. In one example, the thing provided is furnished, supplied, allocated or distributed to something else. “Providing,” refers to an action to provide the thing.

As used herein, “proximate,” means close to or very near.

As used herein, “removed,” with reference to washing a solid support so that a microbe (e.g., bacteria) not attached to another microbe (e.g., fungus) is removed, refers to displacing microbes that have not formed an association with another microbe (e.g., displacing bacteria that have not associated with a fungus). In one example, “removing” may be performed by applying a liquid to a support, onto which a fungus has been established, so that bacteria not attached to the fungus are no longer present on the fungus or the support.

As used herein, “screening,” means an action to evaluate, ascertain or check.

As used herein, “spore,” with reference to bacterial or fungal spores, means an environmentally-resistant form of a bacterium or fungus. Generally, vegetative cells become spores by a process called sporulation. Spores (not capable of dividing) generally become vegetative cells (capable of dividing) by a process called germination.

As used herein, “secondary characteristic,” generally with reference to one or more characteristics of a microbe, means a characteristic that is not growth or facilitation of growth.

As used herein, “solid,” refers to a state of matter that possesses structural rigidity and resistance to changes in shape or volume. Example solids include crystalline solids (e.g., metals) and amorphous solids (e.g., glass).

As used herein, “soluble phosphate,” refers to salts containing phosphorus that are water soluble.

As used herein, “stimulate,” means to increase or activate. “Stimulating,” refers to an action that increases or activates.

As used herein, “supplying,” refers to an action to make something available.

As used herein, “support,” with reference to a solid or semi-solid support, means something that serves as a foundation and/or bears the weight of a thing. Herein, one example of a solid support is a glass cover slide. Herein, one example of a solid or semi-solid support is an agar plate for culturing microorganisms.

As used herein, “testing,” refers to an action to appraise, assess or study.

As used herein, “usable phosphate,” refers to forms of phosphates that can be used by plants. Usable phosphates generally are a subset of inorganic phosphates. Example plant-usable phosphates are hydrogen phosphate and dihydrogen phosphate. Usable phosphates are generally water soluble.

Methods for Detecting/Isolating Microbes that Form Associations

Disclosed herein are methods for identifying/isolating microbes that associate with one another. Generally, the microbes that associate with one another are different from one another (e.g., different species, different genus, and the like). The methods may be used to identify and/or isolate combinations of different associating bacteria, archaea, protozoa, fungi, algae, and the like. In one example, “different from one another,” when referring to an association of microbes, may mean bacteria that associate with archaea, bacteria that associate with protozoa, bacteria that associate with fungi, bacteria that associate with algae, archaea that associate with protozoa, archaea that associate with fungi, archaea that associate with algae, protozoa that associate with fungi, protozoa that associate with algae, fungi that associate with algae, and the like. In one example, “different from one another,” may mean that one species or strain of bacteria associates with another species or strain of the bacteria, one species or strain of archaea associates with another species or strain of the archaea, one species or strain of protozoa associates with another species or strain of the protozoa, one species or strain of fungi associates with another species or strain of the fungi, one species or strain of algae associates with another species or strain of the algae, and the like. The associations may be between two different microbes, three different microbes, four different microbes, five different microbes, and so on. In one example, the association is between a bacterium and a fungus. In one example, the fungus is a non-mycorrhizal fungus.

Example terms that may be used to describe the association of a first microbe with a second microbe may be terms like, “associates with,” “attaches to,” “binds to,” and the like. In one example, the “association” between different microbes may be an association empirically defined by a method used to detect the associated microbes. In one example of a method, different microbes are contacted with one another such that associations between different microbes are capable of occurring. In one example, the different microbes may be contacted in liquid. In one example, the different microbes may be contacted on a support. In one example, the conditions under which the microbes are contacted may be close-to-natural conditions. Then, the different microbes that have formed an association may be identified and/or isolated. In one example, microbes that have associated may be identified and/or isolated using one or more selective or enrichment procedures (e.g., antibodies, media, and the like). In one example, it may be possible to select or enrich for associations of certain microbes based on one or more characteristics, properties, functions, and so on, either present in the association or absent from an association.

In one example method, microbes that have not associated with a different microbe may be removed in the methods, so that associated microbes may be more easily identified. In one example, the non-associated (or more weakly associated) microbes may be removed by washing them away, leaving the associated (or more strongly associated) microbes. The removal process may remove some or all of the non-associated microbes. Multiple steps of removal may be performed. In one example, the removal and/or multiple steps of removal may be described as enriching a population of microbes for microbes that have associated with a different microbe. Other methods may be used to identify and/or isolate microbes that associate with each other.

In one example, associations between different microbes may be strong or weak, or in between. In other words, the associations may have different strengths or affinities. For example, one bacterium may associate with a fungus with higher affinity than another bacterium may associate with a fungus. In one example, the affinity of one microbe for another microbe may be described using a binding constant. In one example, the binding constant may be estimated by dividing the concentration of the associated microbes by the product of the concentrations of the individual unassociated microbes. In one example, the affinity of one microbe for a second microbe may be described in absolute terms. In one example, the affinity of one microbe for a second microbe may be described relative to binding of, for example, a third microbe for a fourth microbe. In one example, atomic force microscopy may be used to estimate affinity of microbes for one another.

It may be possible to design the identification/isolation assays, described above, to favor detection of microbe associations with higher or lower affinities. For example, using the washing methods described above to remove microbes, multiple washing steps may dissociate bacteria that have weakly associated with fungi, while not dissociating bacteria that have strongly associated with the fungi. It may be possible, for example, to modify the composition of the liquid/fluid used in the washing procedures (e.g., ionic strength) to favor identification/detection of microbial associations that have certain affinities.

The terms used to describe the association between different microbes, or the term “association” for that matter, does not imply a biological/chemical mechanism by which the association may occur. In one example, an association of two or more microbes may occur, at least in part, because of nonspecific interactions. In one example, an association of two or more microbes may occur, at least in part, because of specific interactions. Some interactions may involve a ligand on one microbe that binds to a receptor on another microbe. Some associations may involve chemical bonding (e.g., ionic bonds or attractions). In one example, interactions may be covalent. In one example, a microbe that associates with a second microbe may colonize the second microbe.

Methods to detect microbial associations may be designed in various ways. For example, a single microbe or type of microbe may be used as “bait” for detecting microbes that may associate with that particular microbe. Also, a population of different microbes may be used as “bait” for detecting associating microbes. The identity of the single microbe, or the identities of some or all of the microbes in a population of microbes used as “bait” may be known or unknown. That is, the methods may be used to identify microbes capable of forming associations with one or more known microbes. The methods may be used to identity microbes capable of forming associations when the identities of the microbes are not known.

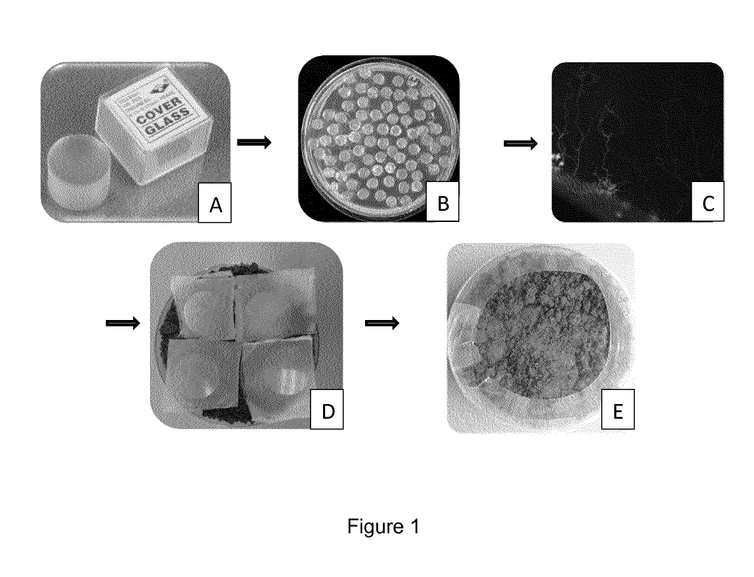

In general, the methods disclosed herein for detecting and/or isolating microbial associations use conditions that are close-to-natural conditions. That is, the aspiration is to make the individual conditions of the laboratory-based method, as well as the laboratory-based method as a whole, the same as or nearly the same as the natural system that the laboratory system is designed to mimic. By using the same conditions as in the natural system, it may be that associations of microbes that are identified and/or isolated using the disclosed methods are the same as those that exist in the natural system. The methods may be performed in liquid or, at least in part, on a surface or support. In one example of the soil-based system disclosed herein, a chosen fungus is inserted into a soil sample (FIG. 1), where the soil sample is, as much as possible, undisturbed or unaltered as compared to the soil from which the sample was obtained. For example, the diversity of microbes within the sample, the temperature, humidity, chemical composition, and the like, are generally unchanged from the soil as it exists in its natural state. While not every possible condition of an environmental sample, as it exists in nature, may be retained or preserved in the close-to-natural situation in the laboratory, as many conditions as possible are sought to be unaltered. The more conditions that are preserved, the more likely will be that the laboratory situation reflects the situation as it exists in nature. In some instances, however, it may be possible to intentionally alter one or more conditions of the sample, in order to favor or disfavor certain microbial interactions.

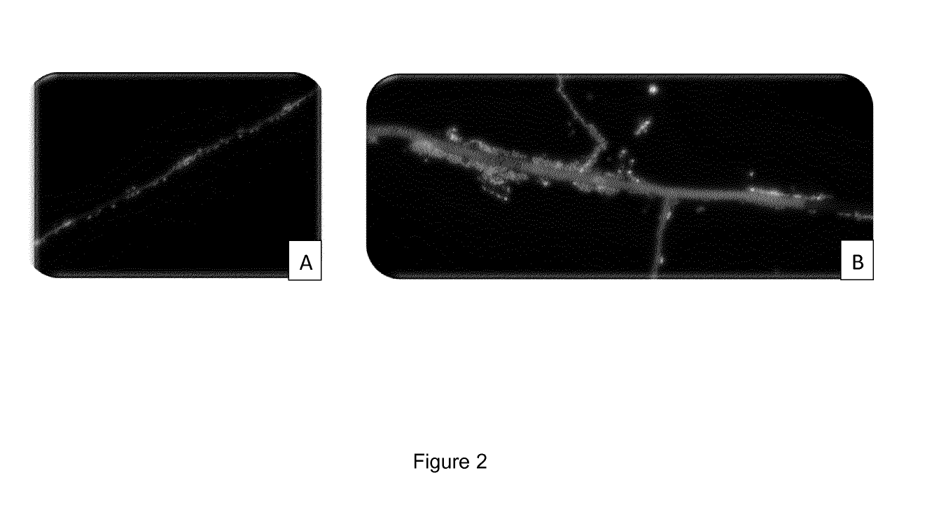

In one example of a method/assay, a non-mycorrhizal fungus, may be established on a support (e.g., glass slide, cover slip, polycarbonate filter, and the like). The support, with the established fungus, may be exposed to an environment that may contain microbes that are capable of associating with the fungus. Or, for example, the established fungus may be exposed to an environment in which it is not known whether microbes capable of associating with the fungus are contained therein. In one example, the environment may be a soil-based environment (e.g., a soil microcosm). In one example, the fungus may be exposed to a soil microcosm by placing the support on which the fungus is established into a mesh bag, which then is placed into the soil microcosm (FIG. 1). In one example, the mesh bad is not used. The mesh may allow microbes to pass through, so that the microbes in the soil can contact the support and the fungus established on the support. The support may be kept in the microcosm for various periods of time (e.g., days). For example, the support may be kept in the microcosm for multiple days (e.g., 1, 2, 3, 4, 5, 6, 7, 8, 9, 10, 11, 12 or more days). After that time, the support may be removed from the soil microcosm and washed, to remove microbes that have not associated, or have weakly associated, with the fungus established on the support. FIG. 2 shows an example micrograph of bacteria that have formed associations with hyphae of the fungus, Penicillium bilaiae, after use of this method.

In one example, the fungi used as “bait” to detect bacteria capable of forming associations with the fungi are non-mycorrhizal fungi. Example non-mycorrhizal fungi may include, but are not limited to, fungi from the genera Aspergillis, Fusarium, Alternaria, Achrothicium, Arthrobotrys, Penicillium, Cephalosporium, Cladosprium, Curvularia, Cunnighamella, Candida, Chaetomium, Humicola, Helminthosporium, Paecilomyces, Pythium, Phoma, Populospora, Myrothecium, Morteirella, Micromonospora, Oideodendron, Rhizoctonia, Rhizopus, Mucor, Talaromyces, Trichoderma, Torula, Schwanniomyces and Sclerotium. In one example, the non-mycorrhizal fungi used may be capable of solubilizing one or both of organic and inorganic phosphate.

Example non-mycorrhizal fungi may include, but are not limited to, the following fungi: Arthrobotrys oligospora, Aspergillus awamori, Aspergillus niger, Aspergillus tereus, Aspergillus flavus, Aspergillus nidulans, Aspergillus foetidus, Aspergillus wentii, Fusarium oxysporum, Alternaria teneius, Penicillium digitatum, Penicillium lilacinium, Penicillium bilaiae, Penicillium funicolosum, Penicillium aculeatum, Curvularia lunata, Chaetomium globosum, Humicola inslens, Humicola lanuginosa, Paecilomyces fusisporous, Populospora mytilina, Myrothecium roridum, Rhizoctonia solani, Trichoderma viridae, Torula thermophila, Schwanniomyces occidentalis and Sclerotium rolfsii. In one example, the non-mycorrhizal fungi used may be capable of solubilizing one or both of organic and inorganic phosphate. In one example, fungi used in the methods may be ascomycetes.

Although the disclosed methods may be used for identifying and/or isolating, from a population of different microbes (e.g., a heterogeneous or diverse population), individual microbes that can form associations, there are other ways to use the methods. In one example, the methods may be used as a type of strain enrichment or selection procedure, or a procedure for identifying/isolating variants or mutants from a pure or clonal population of microbes. For example, one could start with Penicillium bilaiae, established on a cover glass as already described, and then contact the cover glass with a population containing a single strain of bacterium, under conditions where associations of the bacterium and Penicillium bilaiae are possible to occur. In one example, the population of the single strain of bacteria may be mutagenized before contacting the cover glass with the bacteria. In one example, the population of a single strain of bacterium may be a bacterium that is known to form associations with Penicillium bilaiae, in which case the procedure is an enrichment- or selection-type procedure, in that individual microbes from the population that have an increased ability to form associations are sought. In one example, the population of a single strain of bacterium may be a bacterium that is not known to associate with Penicillium bilaiae, in which case the procedure seeks individual microbes from the population that have acquired the ability to form associations with Penicillium bilaiae. The methods may employ conditions, for example, to enrich for variants or mutants within the pure bacterial population, where the variants or mutants have or have an increased capability to associate with or to stay associated with the fungus. In one example, after associations of the bacteria and fungus have occurred, it may be possible to repeatedly wash the cover slide on which the fungus has been established, to dissociate all but a subset of the bacteria that bind to Penicillium bilaiae with high affinity. Higher-affinity bacterial binders may be identified using these methods or variations thereof.

In one example, these strain enrichment/selection or variant/mutant isolation procedures may be used to identify/isolate Bacillus amyloliquefaciens that associate/associate better with Trichoderma virens fungi. In one example, the Bacillus amyloliquefaciens strains may be FZB24 or ATCC BAA-390; the Trichoderma virens strains may be ATCC 58678 or G1-21, as disclosed in U.S. Pat. No. 7,429,477 (Ser. No. 10/940,036), issued 30 Sep. 2008.

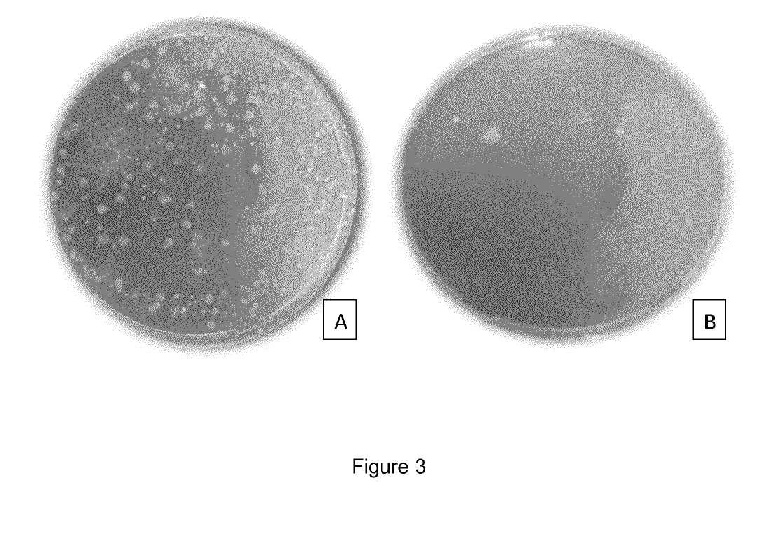

Once microbial associations have formed, the microbes forming the associations may be identified and/or isolated. In one example, one or more microbes forming an association may be cultured. FIG. 3 shows an example of culturing bacteria that have formed associations with the fungus, Penicillium bilaiae. FIG. 3A shows colony forming units (CFU) of bacteria obtained by scraping Penicillium bilaiae from cover slides, after the cover slides were exposed to a soil microcosm, and plating the scrapings. These colonies of bacteria represent example bacteria that have formed associations with the fungus. FIG. 3B shows CFU of bacteria obtained from a control experiment, where cover slides onto which Penicillium bilaiae were not established, were treated similarly. Generally, a pure culture of a bacterium that has been isolated due to its ability to associate with a fungus is capable of colonizing the fungus to which it associated with when the pure culture of bacteria is inoculated together with the fungus.

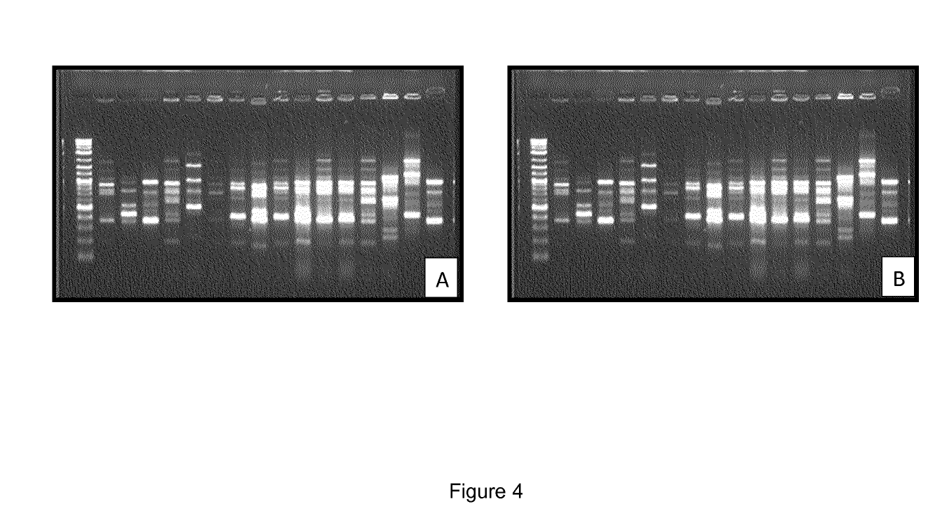

Microbes identified as forming associations with other microbes may be characterized by methods known in the art. In one example, genomes of some bacteria (e.g., 200 isolates) identified as associating with Penicillium bilaiae may be characterized by a genomic fingerprinting method called UP-PCR, as described in Example 1 and illustrated in FIG. 4. Herein, UP-PCR indicated that the 200 isolates represented 156 different UP-PCR groups. In one example, 16S rRNA/rDNA sequencing may be used to obtain at least partial 16S rRNA sequences from the microbes, and the sequences may be used to query one or more sequence databases to identify sequences in the database that are related to the query sequence. If identities of organisms from which the 16S sequences in the databases originated are known, it may be possible to identify the organisms in the association, from which the query sequences were obtained. In one example, whole-genome sequencing of genomes from organisms from microbe associations may be used similarly. Example 1 herein is illustrative.

### Screening/Examining for Effects of Associated Microbes on Microbe Growth

Also disclosed are methods for screening microbes for effects on growth of other microbes. In one example, microbes found to form associations with one another may be examined for capability to affect growth of other microbes in the association. In one example of an association of two or more microbes, one microbe in the association may affect growth of a second microbe in the association (e.g., one-way). In one example of an association, a first microbe in the association may affect growth of a second microbe in the association, and the second microbe in the association may affect growth of the first microbe in the association (e.g., two-way). Examples of three-way, four-way, five-way, and so on, effects on growth may be envisioned. A microbe in an association may positively affect, or stimulate/facilitate/promote, growth of second microbe in the association. A microbe in an association may not affect, or may have a neutral effect on growth of an associated microbe. A microbe in an association may negatively affect, or impede, growth of a second microbe in the association. A first microbe that impedes growth of a second microbe may indicate that an association of the two microbes is not a compatible or stable association. One might anticipate that a first microbe that associates with a second microbe, and kills or severely impedes growth of the second microbe, may not be a microbe that is identifiable in an assay designed to detect associations of microbes.

Different characteristics of microbe growth can be measured, and may facilitate a determination of whether one microbe in an association affects growth of a second microbe in an association. In various examples, the doubling time of a microbe during the exponential phase of growth may be measured. The density to which a microbe grows (e.g., in the stationary phase of growth) may be measured. The lag time of a microbe, after inoculation of a culture, before the exponential phase of growth begins, may be measured. The size of a colony of a microbe on a solid or semi-solid medium may be measured. The biomass of organisms may be measured. In one example, growth of a microbe in an environment containing different concentrations of oxygen may be measured. In one example, the capability of one microbe to affect sporulation, or germination of spores, of another microbe may be a method of measuring growth. In one example, an extract from one microbe may be made and its effect on growth of a second microbe may be tested.

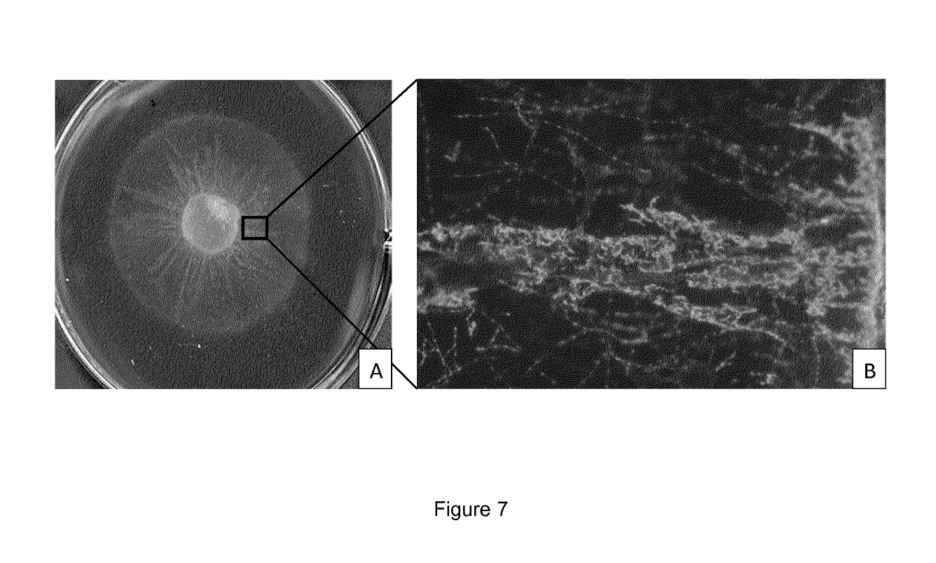

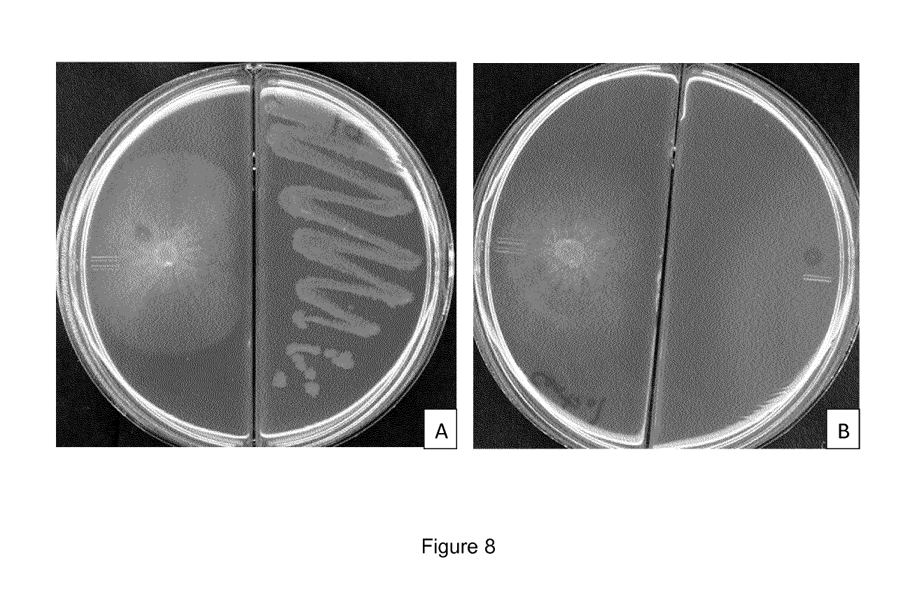

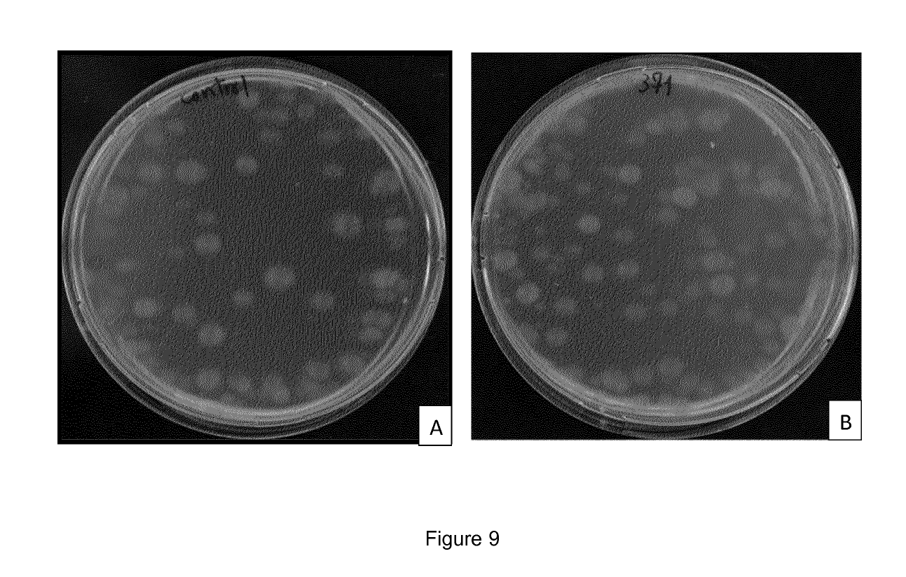

These and other measurements of effects of bacteria on growth of a fungus are illustrated herein. Example results of tests of bacterial strains on growth of Penicillium bilaiae, as measured by size of Penicillium bilaiae colonies, are described in Example 4, and shown in rows 1-4 of Table 2. Example results of tests of volatiles from bacterial strains on growth of Penicillium bilaiae are described in Example 5, shown in row 5 of Table 2, and illustrated in FIG. 8. Example results of tests of bacterial strains on germination of Penicillium bilaiae spores are described in Example 6, shown in rows 6-7 of Table 2, and illustrated in FIG. 9. These bacterial strains (i.e., strains 313, 346, 351, 365 and 371) were also confirmed to associate with/colonize Penicillium bilaiae, as shown in Example 3 and illustrated in FIG. 7.

Other parameters of growth, and methods for quantifying those parameters, exist and may be used. A number of other assays are known in the art for determining an effect of a substance, or another organism, on growth of a microbe. In various examples, effects on growth of a microbe may be examined using molecular techniques, reverse transcription quantitative polymerase chain reaction (RT-PCR) for example, to measure levels of gene products that may correlate with growth. Other chemical components, specific to an organism, for example, may also be measured (e.g., phospholipid-derived fatty acids).

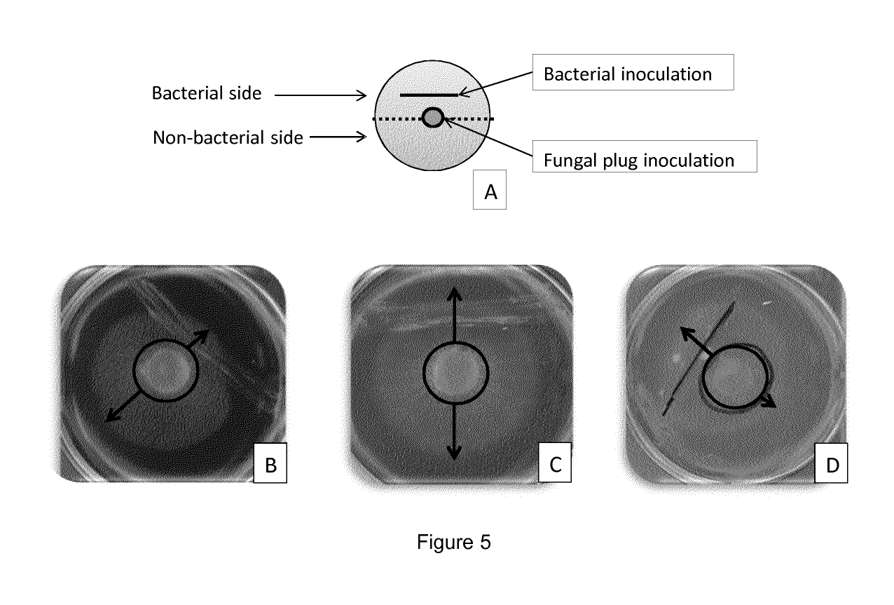

These “growth” assays may be performed on different types of media. The media used in growth assays may contain different types and/or concentrations of nutrients. The media used may contain high concentrations of nutrients. The media used may contain low concentrations of nutrients, or even no nutrients at all. In one example, the medium used in the assays may contain a threshold of types and levels of nutrients, above which, detection of an effect of one microbe on growth of a second microbe may be difficult to detect. In one example, the nutrients used in a growth assay may have no more nutrients than are provided by an amount of agar in the media that provides for a solid or semi-solid medium. For example, 0.25%, 0.5%, 1%, 1.5%, 2.0%, 2.5%, 3.0%, 3.5%, 4.0%, 4.5% or 5.0% agar may be used. Example ranges of agar that may be used includes 0-0.25%, 0-0.5%, 0-1.5%, 0-2.0%, 0-2.5%, 0.25-1.0%, 0.25-1.25%, 0.25-1.5%, 0.25-2.5%, 0.5-1.0%, 0.5-1.5%, 0.5-2.0%, 0.5-2.5%, 1.0-1.5%, 1.0-2.0%, 1.0-2.5%, and the like. The agar may be a standard bacteriological-grade of agar. In one example, the idea of a threshold level of nutrients, above which detection of effects on growth may become difficult, is generally consistent with using close-to-natural conditions to identify associations of microbes and characteristics that the associations may possess. For example, it is believed that, for the assay described in Example 2 and illustrated in FIG. 5, no nutrients/nutrient levels above those present in medium that contains about 1.5% agar will yield good results. Of course, the “threshold level” type and level of nutrients for use in growth-type assays may vary depending on, for example, the type of microbiome the system is intended to mimic. This level may be empirically determined for different applications of the specific growth-type assay that is being implemented.

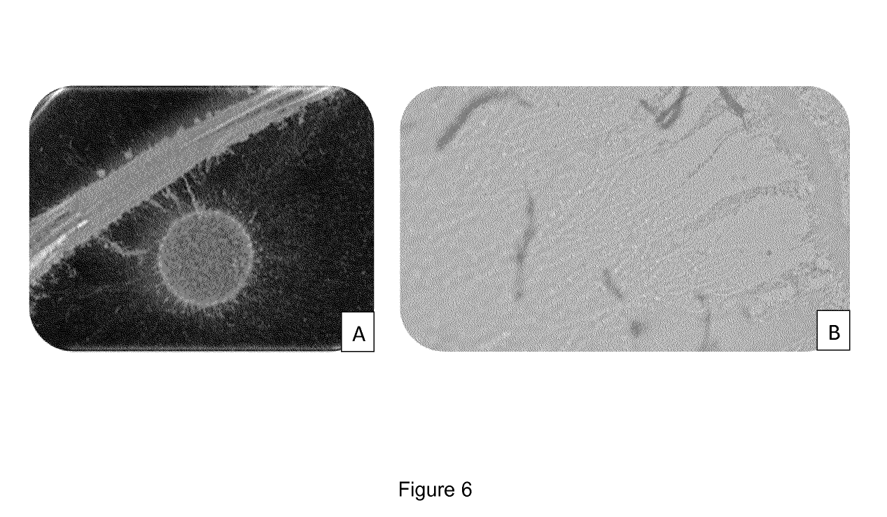

In an example of one method, where the capability of a bacterium to affect growth of a fungus is measured, an assay of the type described in Example 2 and illustrated in FIG. 5, may be used. In one example of this assay, a solid or semi-solid medium (e.g., containing agar), called water agar, is used that contains about 1.5% agar and, generally, no additional nutrients. In this assay, mycelia of a fungus are grown in proximity to a bacterium, on the same petri dish containing the water agar medium. Size and/or symmetry of the mycelial colony may be observed or measured, as compared to control petri dishes that contain fungal mycelia but not bacteria. An effect of the bacteria on growth of the fungus (e.g., positive, negative, neutral) may be determined based on visual inspection, as illustrated in FIG. 5. In one example, a bacterium and a fungus may be placed on a support so they do not initially contact one another and, after a period of time, it is determined whether the bacterium and fungus contact one another, as illustrated in FIG. 6. Herein, 200 bacteria that were isolated as forming associations with Penicillium bilaiae were tested for capability to affect growth of the fungus. The results were that 19% of the bacteria had a positive effect on growth of Penicillium bilaiae, 56% of the bacteria had a negative effect on growth of the fungus, and 19% were neutral.

### Testing for Effects of Microbes on Secondary Characteristics of Associated Microbes

Also disclosed is that, in an association of microbes in which at least one microbe in the association is capable of affecting the growth of another microbe in the association (e.g., one-way, two-way, etc., as described above), some of these associations of microbes may be capable of characteristics that are not an effect of one microbe on the growth of another. Such “secondary” characteristics are characteristics that are not found in any of the microbes that make up the association of microbes, when those microbes are not in association with one another (i.e., when the microbes are “alone”). Therefore, as disclosed herein, an association of microbes, where a primary characteristic or property of the association is an effect of at least one microbe on growth of the other, may produce one or more secondary characteristics or properties not present in the microbes alone. The concept is that, in one example, microbial associations in which a first microbe has an effect on growth of a second microbe, may be associations which possess characteristics that are new or improved compared to characteristics possessed by either microbe alone.

In one example, a secondary characteristic, found in or produced by an association of microbes, as described above, may be a characteristic that did not exist, at least at the limit of detection methods, in any of the individual microbes of the association, when the microbes are alone. For example, in a bacterium that associates with a fungus, the ability of either microbe alone to solubilize phosphate may not be detectable. But, when the bacterium and fungus are “associated” (e.g., co-cultured), an ability to solubilize phosphate, or an activity that solubilizes phosphate may be detected. In such a situation, the characteristic of phosphate solubilization may be said to be a “new” characteristic or activity.

In another example of a bacterium that forms an association with a fungus, one or both of the bacterium and fungus may display detectable activity to solubilize phosphate when alone or not in the association. But, when the bacterium and fungus are “associated,” the level of phosphate solubilization activity may be different than the activities of the two microbes alone. In one example, the activity produced by the association of microbes may be an activity that is additive of the activities of the bacterium and fungus. In one example, the activity produced by the association of microbes may be synergistic (e.g., greater than the sum of activities produced by the bacterium and fungus alone). In the synergistic situation, the characteristic of phosphate solubilization may be said to be an “improved” characteristic or activity. Or, the activity produced by the association of microbes may be antagonistic (e.g., lesser than the sum of activities produced by the bacterium and fungus alone). In such a case, the characteristic of phosphate solubilization may be said to be a “degraded” characteristic or activity.

A wide variety of secondary characteristics of the microbial associations—in fact, at least any characteristic or property that any microbe (not just those in the particular association being investigated) may be known to possess—may be investigated. Secondary characteristics may include almost any characteristic or property of a microbe that has the capability to be measured or estimated. For example, the microbial associations may be tested for capability to produce certain enzymes, bioactive metabolites, signal molecules, various activities, gene products and the like. The microbial associations may be tested for biostimulant activities, nutrient activities, pesticidal activities, plant growth promoter activities, and the like. In one example, the microbial associations may be tested for capability to facilitate plant growth. In one example, secondary characteristics may include presence of, or increase in, levels of activities that inhibit or inhibit one or more bacteria, fungi, insects, mites, nematodes, rodents, snails, weeds, viruses, or other pests, pathogenic or nonpathogenic.

In one example, secondary characteristics may include activities that inhibit or kill a plant pathogen (e.g., biocontrol activities) (e.g., in a soil environment), activities that provide or increase the amount of plant-usable nutrients (e.g., in a soil environment), activities that improve viability of one or more microbes (e.g., under stress conditions), and the like.

In one example, the microbial associations may be tested for the capability to solubilize phosphate. Plants generally require phosphate. Generally, soils contain phosphates, but much of it is insoluble and/or not in a form that can be used by plants. Certain microbes, including some Penicillium bilaiae strains, are able to facilitate solubilization of insoluble phosphate forms in the soil and increase the levels of phosphate in soil that is usable by plants. The ability of certain microbes to solubilize phosphate may correlate with capability of the microbes to facilitate plant growth. However, an association of microbes may facilitate plant growth without solubilizing phosphate (e.g., biocontrol activity).

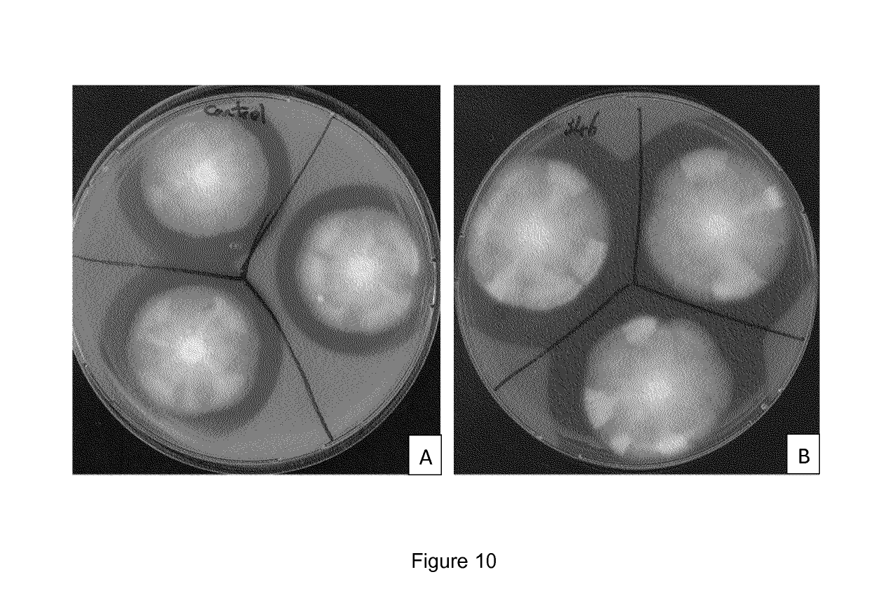

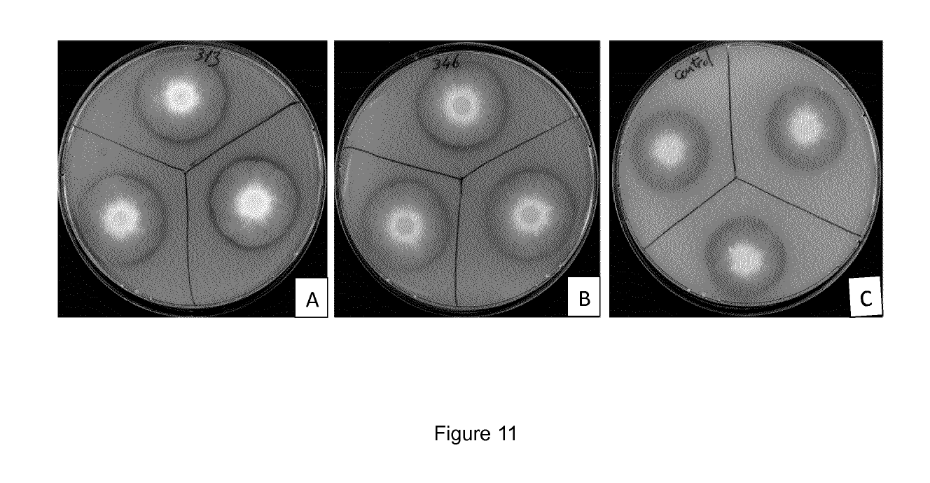

Herein, the 5 bacterial strains that were best able to facilitate growth of Penicillium bilaiae, based on water-agar assays (Example 2 and FIGS. 5 and 6), were tested, in association with Penicillium bilaiae, for capability to solubilize phosphate, as compared to the capability of Penicillium bilaiae alone to solubilize phosphate. Example results of experiments indicating phosphate solubilization as a secondary characteristic that is an “improved” characteristic produced by the association of one of bacterial strains 313 (DSM 32170), 346 (DSM 32171), 351 (DSM 32172), 365 (DSM 32173) and 371 (DSM 32174) with Penicillium bilaiae are disclosed herein. Example results showing that the association of individual of these bacterial strains and Penicillium bilaiae solubilize organic phosphate are described in Example 7, shown in row 8 of Table 2, and illustrated in FIG. 10. Example results showing that the association of individual of these bacterial strains and Penicillium bilaiae solubilize inorganic phosphate are described in Example 8, shown in row 9 of Table 2, and illustrated in FIG. 11.

Generally, these bacteria (strains 313, 346, 351, 365 and 371) in association with Penicillium bilaiae, were better able to solubilize phosphate than Penicillium bilaiae alone (Table 2, rows 8 and 9). Generally, these results are synergistic, because none of the 313 (DSM 32170), 346 (DSM 32171), 351 (DSM 32172), 365 (DSM 32173) and 371 (DSM 32174) strains had detectable phosphate solubilization activity alone, and the activities produced by the association (bacterium+Penicillium bilaiae) was greater than that produced by the Penicillium bilaiae strain alone.

In the example disclosed herein, out of 200 strains of bacteria that were isolated as forming associations with Penicillium bilaiae, 19% of those bacteria (about 38 strains) had a positive effect on growth of Penicillium bilaiae. From those 38 strains, the 5 strains that were most capable of facilitating growth of the fungus were tested for the capability to increase phosphate solubilization of Penicillium bilaiae. All 5 strains (5/38=13%) generally increased the phosphate solubilization of Penicillium bilaiae. At least 4 of the strains (4/38=10%) solubilized an amount of phosphate that was significantly increased as compared to the amount of phosphate solubilized by Penicillium bilaiae alone (Table 2, rows 8 and 9). Therefore, at least in this example, 10% of bacterial strains shown both to form an association with Penicillium bilaiae and also to facilitate growth of Penicillium bilaiae were also able to increase the amount of phosphate that the fungus could solubilize.

### Compositions

The compositions herein are generally compositions that contain one or more microbes. For example, compositions containing less than the full complement of microbes that make up an association of microbes may be designed to be combined or mixed at some point (e.g., immediately before use) so that the full complement of different microbes that make up an advantageous microbial association is present. In one example, a composition of a specific microbe may be designed or formulated so that viability of the microbe in the composition is retained for as long a period as possible (e.g., days, weeks, months or years). Such a composition, that optimally facilitates survival/viability of one microbe, may not optimally facilitate survival/viability of a second microbe. Therefore, separate compositions may be designed for maximal viability of different microbe components of a microbial association, and the separate compositions may also be designed to be mixed or combined before use. However, in some cases, it may be possible to design single compositions that can contain all of the different microbes of an advantageous microbial association. The compositions disclosed herein may be solid compositions (generally water soluble) or liquid compositions.

The microbe compositions may contain a variety of components in addition to microbes. These additional components may be naturally occurring or may not be naturally occurring (e.g., synthetic). Even if one or more of the additional components are naturally occurring, they may be combined with one or more other naturally-occurring components, or with synthetic components, to yield a composition that is not naturally occurring. The combination of these additional components, naturally occurring, not naturally occurring, or naturally occurring mixed with non-naturally occurring may, in combination, may provide advantages to the composition as a whole that are significant. For example, there may be combinations of these components that, for specific microbes, are superior in retaining viability of the microbe over a period of time, that prevent contamination of the compositions by unwanted microbes, or that have other functions. In one example, a microbe composition may contain one or more additional components that are excipients. In one example, an excipient may be naturally occurring. In one example, an excipient may be non-naturally occurring (e.g., synthetic).

In one example, an excipient that is used may be an antimicrobial agent. Such an agent may be used to prevent contamination of the composition with one or microbes other than those that are part of the desired microbial association. Generally, a specific type of antimicrobial agent (e.g., an antifungal agent) might be expected to be used in compositions that do not contain that specific type of microbe (e.g., fungi). However, for example, certain antifungal agents may be used in compositions of fungi if, for example, the agent is fungistatic rather than fungicidal. It may also be possible to use a concentration of an antifungal agent that is diluted to a level below an effective level when combined with another composition or when used.

In one example, an excipient that is used may be a spore anti-germinant. In some compositions, the microbes contained therein may be in the form of spores. Generally, spores of both bacteria and of fungi, are more resistant to certain environmental conditions that are the vegetative forms of bacteria and fungi. In compositions that contain spores, the ability of the spores to remain as spores (i.e., for the spores not to germinate to become vegetative cells) is generally desirable. Therefore, one or more anti-germinants may be added to the compositions. Generally, concentrations of anti-germinant substances used in these compositions are such that they become diluted to concentrations that are ineffective or inactive when the compositions are mixed with other compositions or when used. The microbial compositions herein generally contain concentrations or amounts of microbes that are effective for an intended purpose. In one example, concentrations of microbes in the compositions are higher than concentrations of the microbes found in nature. In one example, concentrations of the microbes may be in the range of 5×10⁷, 1×10⁸, 5×10⁸, 1×10⁹, 5×10⁹, 1×10¹⁰, 5×10¹⁰ or 1×10¹¹ organisms per gram of water-soluble solid or per milliliter of the liquid. In one example, the compositions may contain at least 50%, 60%, 70%, 80%, 90%, 95% or 99% of the organisms in the compositions as spores.

Compositions containing microbes may also contain one or more components from the medium in which the microbes were propagated. For example, when microbes are produced in large quantities or volumes, they may not be purified away from all components of the medium in which they were grown. In one example, bacteria that are fermented in large volumes may be concentrated/dried using a process called spray drying. Under some circumstances, the spray-dried products contain dried microbial spores, for example, along with levels of media components from the fermentation medium. In some cases, because at least some components of the fermentation medium may not be naturally occurring, the spray-dried product also contains non-naturally occurring components.

In one example, the compositions contain one or more microbes isolated using methods disclosed herein. In one example the microbes may include one or more of the bacterial strains 313 (DSM 32170), 346 (DSM 32171), 351 (DSM 32172), 365 (DSM 32173) and 371 (DSM 32174) disclosed herein. The identities of these bacterial strains are described in Table 1, in Example 2. The data indicate that these strains are from the genus Bacillus. As such, these strains may sporulate to form spores. In one example, compositions containing one or more of these strains also contain Penicillium bilaiae, or are designed to be combined or mixed with compositions containing Penicillium bilaiae.

One or more of the compositions may be part of a kit. The kit may contain containers configured to house one or more of the microbes of an association. The compositions, whether or not part of a kit, may be marketed. Marketing may include one or more of advertising, promoting, storing, offering for sale, selling, distributing, shipping, and the like, of one or more of the compositions. Generally, the compositions may be marketed for a use. In one example, the use may be for providing usable phosphate to a plant. In one example, the use may be for facilitating growth of a plant.

Example compositions containing part or all of the microbial associations, or combinable with part of all of the microbial associations, may contain other ingredients or substances. In one example, the compositions may be combined with one or more plant signal molecules including but not limited to, lipo-chitooligosaccharides (LCOs), chitooligosaccharides (COs), chitinous compounds (e.g., chitins, chitosans), flavonoids (e.g., daidzein, genistein, hesperitin, naringenin, lutiolin), jasmonic acid or derivatives thereof, linoleic acid or derivatives thereof, linolenic acid or derivatives thereof, karrikins nutrients (e.g., vitamins, macrominerals, trace minerals, organic acids, various elements), gluconolactones, glutathiones, biostimulants, and the like.

Example compositions may also contain one or more microbes not identified by the methods disclosed herein. The other microbes may have one or both of biocontrol and inoculant properties. Also, suitable acaricides, fungicides, gastropodicides, herbicides, insecticides, nematicides, rodenticides, virucides, and the like could be contained in the compositions.

The compositions may also contain substances such as microbial extracts, natural products, plant defense agents and the like.

### Methods for Using the Compositions

In one example, the microbes isolated using the methods disclosed herein, may be used for specified purposes, generally in combination with the fungi to which they associate (e.g., Penicillium bilaiae strain P-208, or strains P-201 and P-208). In one example, the associations of microbes disclosed herein, and the compositions containing these microbes may be used to facilitate plant growth. A variety of plants may be used. In one example, the compositions may be used to facilitate growth of plants that use phosphate. In one example, compositions of microbes may be used to facilitate growth of plants such as wheat, peas, chickpeas, lentils, lupins, faba beans, canola, sorghum, corn, soybeans, and other plants. Example plants may also include, without limitation, oil seed rape, maize, barley, canola, and the like.

Facilitation of plant growth may be measured by determining increases in a variety of parameters, including increase in plant yield, for example. Other parameters of plant growth that may be measured may include, for example, biomass of a plant or parts of a plant (e.g., pods) or numbers of pods per plant. The increases that occur when a combination of fungus and bacteria are supplied to a plant may be additive as compared to the increases that occur when a fungus alone or bacteria alone are used. The increases from the combination may be synergistic in that they are greater than additive of the increases that occur when a fungus alone or bacteria alone are used. The increases due to the combination may be 1.1, 1.2, 1.3, 1.4, 1.5, 1.6, 1.7, 1.8, 1.9, 2.0, 2.1, 2.2, 2.3, 2.4, 2.5 or more fold greater than increases due to the bacteria alone or the fungus alone.

Methods by which beneficial microbes are supplied to plants are known in the art. In one example, the microbes may be applied to a seed. This application may take the form of a seed coating. In one example, the microbes may be applied to a furrow in which a seed or seedling is planted. In one example, the microbes may be applied as a foliar application (e.g., sprayed onto a plant).

The compositions of microbes may be supplied to plants along with one or more biostimulants, nutrients, pesticides, plant signal molecules, pesticides, as well as other compounds or components. In one example, pesticides may include acaricides, fungicides, gastropodicides, herbicides, insecticides, nematicides, rodenticides, virucides, and the like.

### EXAMPLES

The following examples are for the purpose of illustrating various embodiments and are not to be construed as limitations.

### Example 1. System for Isolating Bacteria that Attach to Non-Mycorrhizal Fungi

A system for identifying and isolating bacteria from the soil that associate with the hyphae of a non-mycorrhizal fungus, Penicillium, was developed (FIG. 1). Penicillium bilaiae (either strain P-201, deposited as NRRL 50169, or strain P-208, deposited as NRRL 50162) was grown on ⅕ strength potato dextrose agar (PDA) overnight at 26° C. Then, 6 sterile glass cover slides (FIG. 1A) were placed on water agar plates (15 g agar per liter of MilliQ® purified water) and 12 fungal plugs were placed along the border of each slide, and a single fungal plug was placed in the middle of each glass cover slide (FIG. 1B). The plates were incubated at 26° C. for 3 days to establish Penicillium bilaiae growth on the surface of the cover slides (FIG. 1B). This ensured maximal coverage of the cover slides by Penicillium mycelium and minimized damage to the mycelium when they were removed from the cover slides. Fungal hyphae could be viewed on the cover slips after staining with calcofluor-white and viewing under ultraviolet light at 10× magnification (FIG. 1C). The fungal plugs were gently removed from the cover slides and the cover slides were transferred to sterile nylon mesh bags (mesh size generally 40 μm), which were subsequently sealed by heating. A soil microcosm was prepared in petri dishes (FIG. 1D). The mesh bags, containing the cover slides, were transferred to the petri dishes, covered by soil, and incubated at 26° C. for 8 days (FIG. 1E). Control cover slides, without Penicillium bilaiae, were incubated similarly.

After 8 days, the mesh bags were removed from the petri dishes and cover slides removed from the mesh bags. The cover slides were gently washed twice with 500 μl MilliQ® purified water. To visualize the fungal hyphae, and hyphae-colonizing bacteria, the cover slides were stained with SYBR® Green and visualized under a fluorescent microscope. FIG. 2 shows two micrographs of fungal hyphae, showing small particles of more intense staining on the exterior of the hyphae. The particles are bacteria that have attached to the hyphae.

To isolate the bacteria that have attached to the hyphae, 200 μl of MilliQ® purified water was added to a washed cover slide and the fungal hyphae and attached bacteria were scraped from the cover slide with a scalpel. The MilliQ® purified water suspension was transferred to a 1.5 μl tube with a pipet. Serial dilutions of the suspension were made and a 1000-fold dilution was spread onto 1/10 strength Reasoner's 2A agar (R2A) supplemented with 50 μg nystatin per ml of medium to prevent fungal growth. The plates were incubated at 26° C. for 48 hours. FIG. 3 shows example colony counts from cover slides as above, which contained Penicillium hyphae (FIG. 3A), and from control cover slides, that did not contain Penicillium (FIG. 3B). The cover slides that contained Penicillium hyphae had approximately 100-fold higher bacterial counts than did the cover slides that had no hyphae. This result is consistent with the idea that the cover slide/mesh bag system enriched for bacteria that associated with/attached to the hyphae.

Two hundred individual bacteria colonies were selected from the cover slides that contained hyphae and were twice purified by streaking onto new 1/10 R2A plates. To begin to characterize the different bacterial clones, overnight cultures were streaked to yield individual colonies. A single colony representative of each of the 200 isolates was suspended in 3 ml of PBS (pH 7.4). After centrifuging at 10,000×g for 5 min., the supernatant was discarded and the bacterial cell pellet was resuspended in 100 μl of purified water. The cell suspension was boiled for 10 min at 99° C. in a heating block and then immediately transferred to ice. The lysates were stored at −20° C. until use.

The lysates were then used in a universally primed PCR fingerprinting technique (UP-PCR; Lübeck et al., Delineation of Trichoderma harzianum into two different genotypic groups by a highly robust fingerprinting method, UP-PCR, and UP-PCR product cross-hybridization. Mycological Research 103, 289-298, 1999), using the primer 5′GAG GGT GGC GGC TAG-3′. Twenty μl reaction mixtures were prepared containing 2 μl 10×PCR buffer, 100 μM of each of four deoxyribonucleoside triphosphates, 1 ng/μl of PCR primer, 1 μl of cell lysate and 0.5 μl of Taq polymerase. PCR reactions were performed with a GeneAmp® PCR System 9700 using the following program: initial denaturation at 94° C. for 3 min; 32 cycles consisting of denaturation at 94° C. for 60 sec., primer annealing at 53° C. for 60 sec., elongation at 72° C. for 60 sec; a final elongation step of 3 min. was included at the conclusion of the run. PCR products were separated by electrophoresis through a 1.5% agarose gel and band patterns were visualized after staining with GelRed™ and imaging using a Gel Doc 2000 System (Bio-Rad, USA). The UP-PCR band patterns were grouped manually as described by Worm and Nybroe (Input of Protein to Lake Water Microcosms Affects Expression of Proteolytic Enzymes and the Dynamics of Pseudomonas spp. Appl. Environ. Microbiol., 67, 4955-62, 2001). The UP-PCR results (examples shown in FIG. 4) indicated 156 different UP-PCR groups among the 200 isolates.

Partial 16S rRNA sequences were determined for the 156 different UP-PCR groups. Lysates containing DNA obtained from the bacterial isolates as template. Universal PCR primers 27F and 1429R (Lane D J. 16S/23S rRNA sequencing. In: Stackebrandt E, Goodfellow M, editors. Nucleic acid techniques in bacterial systematics. Chichester, United Kingdom: John Wiley and Sons; 1991. pp. 115-175) were used to amplify a 1.4 kb fragment of the 16S rRNA gene. Fifty μl reaction mixtures contained 5 μl 10×PCR buffer, 4μ of 10 mM of total dNTPs, 2.5 mM of both PCR primers, 0.5 U of Taq DNA polymerase (Sigma) and 2 μl of cell lysate. The PCR program consisted of an initial denaturation step at 95° C. for 5 min., followed by 30 cycles of denaturation at 94° C. for 30 sec., primer annealing at 57° C. for 60 sec., elongation at 72° C. for 90 sec; a final elongation step of 10 min. was included at the end of the run. PCR products were separated by electrophoresis through a 1.5% agarose gel and band patterns were stained with GelRed™ and imaging using a Gel Doc 2000 System (Bio-Rad, USA). PCR amplicons were purified using QIAquick® PCR Purification Kits (Qiagen, USA) and sequenced by GATC Biotech (Germany). To identify the bacterial isolates, the obtained sequences were used as query sequences in BLAST identity searches (http://blast.ncbi.nlm.nih.gov/Blast.cgi). A putative bacterial genus was assigned to each partial 16S rRNA sequence based on identity of the query sequence with sequences in the BLAST database. The assignments were as follows: 72% Bacillus, 18% Pseudomonas, 8% Acinobacter, 1% Firmicutes and 1% Arthrobacter.

### Example 2. Screening for Bacteria that Facilitate Growth of Non-Mycorrhizal Fungi

All 200 of the isolated bacteria were tested for their effects on Penicillium growth using a confrontation-type assay. An agar plug (i.e., fungal plug) from an overnight culture of Penicillium bilaiae (either strain P-201, deposited as NRRL 50169, or strain P-208, deposited as NRRL 50162) on ⅕ strength PDA was placed face-down on water agar in 6-well plates. A straight line of a single bacterial clone was streaked at a 2 mm distance from the fungal plug (FIG. 5A). Petri dishes were incubated at 26° C. for one week. Each bacterium was scored as positive, negative or neutral based on their effect on the fungal growth pattern. If fungal radial growth was higher on the bacterial side of the plate as compared to the non-bacterial side, the bacterium was scored positive (FIG. 5C). If the fungus was growth-inhibited on the bacterial side, compared to the non-bacterial side, the bacterium was scored negative (FIG. 5C). If the bacterium did not affect fungal growth (same radial growth on both sides), it was scored neutral (FIG. 5D). Plates inoculated solely with Penicillium bilaiae were used as control.

Microscopic examination of some of the plates that showed a positive effect of a bacterium on radial growth of Penicillium are shown in FIG. 6. These data show physical interactions between the bacteria and fungus, and suggest that, in general, these bacteria exhibit hyphae-colonizing ability.

The results of the screening showed that 56% of the bacteria had a negative effect on fungal radial growth, 19% of the bacteria had a positive effect on fungal radial growth and 25% of the bacteria were neutral in relation to fungal radial growth. Based on this screening, the five bacteria showing the highest growth promotion on the fungus were selected for additional functional testing. The strain designation of the five selected bacteria, as well as their closest culturable relative based on the 16S rRNA BLAST identity searches described in Example 1 are shown in Table 1. In each case, the habitat from which each of the GeneBank closest relatives in Table 1 were isolated is soil. These strains have been deposited at the DSMZ depository on 8 Oct. 2015, as described in Table 1.

TABLE 1
Strain designation and taxonomic relationship, based on 16S rRNA
sequences

| | | GenBank |
|---|---|---|
| Strain | | accession |
| designation | Closest cultured | number of |
| (deposit | relative in the GenBank | closest |
| number)¹ | database (% identity) | cultured relative |
| 313 | Bacillus simplex LMG11160 (100%) | NR_114919.1 |
| (DSM 32170) | | |
| 346 | Bacillus mycoides strain IHB B 6293 | KR233754.1 |
| (DSM 32171) | (100%) | |
| 351 | Bacillus sp. SC119(2010) (100%) | HM566472.1 |
| (DSM 32172) | | |
| 365 | Bacillus sp. SC119(2010) (100%) | HM566472.1 |
| (DSM 32173) | | |
| 371 | Bacillus simplex LMG11160 (100%) | NR_114919.1 |
| (DSM 32174) | | |

¹Deposits were made on 8 Oct. 2015 by the University of Copenhagen at the DSMZ depository (Leibniz Institute DSMZ-German Collection of Microorganisms and Cell Cultures, Inhoffenstraße 7B, 38124 Braunschweig, Germany)

Phenotypic characterization of these 5 strains is described below.

### Example 3. Microscopic Characterization of Fungal Colonization

After the bacterial strains listed in Table 1 were isolated, using the example process described in Example 1, the bacteria were inoculated together with the fungi and the ability of the bacteria to colonize the fungi was examined.

All five of the selected bacterial strains listed in Table 1 showed colonization of hyphae after 8 days of incubation when the bacteria were grown in proximity to the fungi (FIG. 7B). The data indicated that the bacteria colonized and grew along the fungal hyphae, in some parts of the mycelium, but not in others. No bacterial growth was identified on hyphae near the outer edge of the hyphae. It was also observed that bacterial colonization of hyphae increased over a month's time.

All five of the selected bacterial strains listed in Table 1 also showed colonization of fungal hyphae under close-to-natural conditions, here a soil microcosm. Fungal plugs of Penicillium bilaiae, as described in Example 2, were placed face-down on sterile cover slides and incubated at 26° C. on water agar plates. After 3 days, the fungal plugs were removed and the cover slides were transferred to sterile nylon mesh bags, which were sealed by heating. The mesh bags were transferred to petri dishes, and covered with γ-irradiated soil to which 10⁸ bacteria were added per gram of soil. The petri dishes were sealed and incubated at 26° C. After 14 days, the cover slides were removed, washed and stained with SYBR Green and calcofluor-white, and viewed by fluorescence microscopy. Similar to the results above, shown in FIG. 7, it was shown that all five of the bacterial strains colonized the fungi.

### Example 4. Effect of Bacteria on Fungal Growth and Other Fungal Properties

The five bacterial strains were tested for their ability to stimulate fungal growth in various media. Growth testing was performed by mixing 10 μl of a bacterial suspension with 10 μl of Penicillium bilaiae spores (either from strain P-201, deposited as NRRL 50169, or from strain P-208, deposited as NRRL 50162), and the mixture was added to the center of water agar plates (Table 2, row 1), artificial root exudate (ARE) agar plates (50 mM of each of fructose, glucose and sucrose and 2.95 g succinic acid, 3.35 g malic acid, 2.18 g L-arginine, 1.31 g L-serine, 1.97 g L-cysteine and 15 g agar per liter of MilliQ® purified water; Table 1, row 2), Sperber agar (10 g glucose, 0.5 g yeast extract, 0.1 g CaCl₂, 2.5 g Ca₃(PO₄)₂, 0.25 g MgSO₄, 15 g agar, all per liter of purified water; Table 1, row 3), and calcium phytate agar (1.5% Glucose, 0.3% CaCl₂, 0.5% NH₄NO₃, 0.5% KCl, 0.05% MgSO₄, 0.001% FeSO₄, 0.001% MnSO₄, 0.5% phytate and 20 g agar per liter of MilliQ® purified water; Table 1, row 4). Controls were 10 μl of purified water mixed with 10 μl of Penicillium bilaiae spores. The fungal growth diameter (in mm) was measured after 7 days.

The data (Table 2) showed strains 313, 346 and 365 all significantly increased Penicillium bilaiae mycelium diameter as compared to controls, on at least one medium. Strain 365 showed the largest effect on water agar plates. In addition to outgrowth diameter, as indicated in

Table 2,the bacterial strains also generally increased fungal biomass, as estimated by visual inspection.

TABLE 2
Characteristics of Penicillium bilaiae in association with bacterial
strains (each row of values is from a separate experiment)

| | | Bacterial strains¹ | | | | | |
|---|---|---|---|---|---|---|---|
| Row | | Control | | | | | |
| number | Characteristic | (no bacteria) | 313 | 346 | 351 | 365 | 371 |
| 1 | Colony diameter | 33.0 | 34.0* | 34.4* | 33.6* | 34.2* | 33.4 |
| | on water agar² | 22.0 | 24.8* | 23.9* | 24.0* | 23.9* | 23.2* |
| | (mm) | | | | | | |
| 2 | Colony diameter | 24.8 | 27.0* | 26.0* | 26.4* | 25.8* | 25.5* |
| | on water agar + | 30.7 | 30.7 | 31.3 | 30.9 | 31.1 | 30.8 |
| | root exudates² | | | | | | |
| | (mm) | | | | | | |
| 3 | Colony diameter | 25.4 | 30.0* | 30.0* | 25.4* | 27.4* | 26.0* |
| | on Sperber agar² | 25.4 | 30.0* | 30.0* | 25.4* | 27.4* | 26.0* |
| | (mm) | | | | | | |
| 4 | Colony diameter | 33.8 | 35.6* | 36.0* | 34.4 | 35.8* | 34.6 |
| | on calcium | 34.0 | 35.6* | 36.0* | 34.6* | 36.0* | 34.8* |
| | phytate plates² | | | | | | |
| | (mm) | | | | | | |
| 5 | Volatile effect | 43.2 | 44.4* | 45.4 | 45.8* | 46.0* | 43.6 |
| | colony diameter³ | 48.4 | 50.0 | 51.2* | 49.6 | 50.8* | 49.0 |
| | (mm) | | | | | | |
| 6 | Spore germination | 106 | 143* | 152* | 146* | 150* | 137* |
| | on water agar⁴ | 198 | 250* | 259* | 208 | 254* | 201 |
| | (number) | | | | | | |
| 7 | Spore germination | 201 | 251* | 256* | 204 | 253* | 200 |
| | on water agar + | 181 | 237 | 223* | 245* | 245* | 217* |
| | root exudates⁴ | | | | | | |
| | (number) | | | | | | |
| 8 | Clearing zone on | 47.2 | 49.6* | 53.0* | 47.4 | 53.0* | 51.4* |
| | calcium phytate | 47.4 | 49.6* | 53.0* | 47.4 | 53.0* | 51.2* |
| | plates (organic | | | | | | |
| | phosphate | | | | | | |
| | solubilization)² | | | | | | |
| | (mm) | | | | | | |
| 9 | Clearing zone on | 27.3 | 31.7* | 31.7* | 27.6 | 28.4* | 28.6* |
| | Sperber agar | 27.3 | 31.7* | 31.8* | 27.6 | 28.4* | 28.8* |
| | (inorganic | | | | | | |
| | phosphate | | | | | | |
| | solubilization)² | | | | | | |
| | (mm) | | | | | | |

¹Asterisks next to a value indicate treatment averages with a 95% confidence interval not overlapping the 95% confidence interval of the average value for the corresponding control. Therefore, Pₛₐₘₑ for these treatments and corresponding control averages <0.05 and treatments are considered significantly different from controls. Ninety-five percent confidence intervals were calculated for each average, assuming normal distribution of replicate values.
²Values obtained after 7 days incubation in darkness at 25° C.
³Values obtained after 13 days incubation in darkness at 25° C.
⁴Values for number of germinated spores obtained after 4 days incubation in darkness at 25° C.

### Example 5. Effect of Bacterial Volatiles on Fungal Growth

To evaluate the effect of volatile substances from the bacteria on fungal growth, split water agar plates were used (FIG. 8). The bacterial strains were streaked on one side of the “split.” Penicillium bilaiae spores (10 μl) were placed on the other side of the split. Mycelium outgrowth diameter was measured after 7 days. Controls were Penicillium bilaiae spores only—no bacteria were streaked on the plate. Since there is not a continuous layer of agar between the bacteria and the fungus, the effect of bacterial substances that might diffuse through the agar to affect growth of the fungus are thought to be eliminated. Any effect of the bacteria on the fungus is thought to be provided by volatile substances from the bacteria. FIG. 8A shows strain 365 bacteria streaked on the right side of the split and a positive effect on mycelium outgrowth of the fungus, which was placed on the left side of the split. FIG. 8B shows a control plate, where no bacteria were streaked on the plate. Control fungal outgrowth, in absence of bacteria, is shown on the left side of the split plate.

The data (Table 2) show that strain 365 significantly increased Penicillium bilaiae mycelium diameter as compared to controls. In addition to outgrowth diameter, strain 365 also increased fungal biomass, as estimated by visual inspection.

### Example 6. Effect of Bacteria on Fungal Spore Germination

To test the effects of the bacterial strains on germination of Penicillium bilaiae spores, 200 μl of spores (1 spore/μl) and 20 μl of bacterial suspension was mixed and immediately placed on water or ARE agar and incubated at 26° C. The number of germinated spores were counted after 4 days (FIG. 9B). Spores mixed with 0.9% NaCl were used as control (FIG. 9A).

Table 1, row 6, shows results on water agar plates. Table 1, row 7, shows spore germination results on ARE agar plates. The data show that all 5 bacterial strains increased the number of germinated spores as compared to controls that did not have bacteria.

### Example 7. Effect of Bacteria on Organic Phosphate Solubilization by Fungus

The effect of the bacterial strains on the ability of Penicillium bilaiae to solubilize organic phosphate was tested. To test solubilization of organic phosphate, calcium phytate agar plates were used. Five μl of Penicillium bilaiae spores was mixed with 5 μl of bacterial suspension, and this was added as a single drop to the plates and the plates were incubated at 26° C. After 7 days, the zone of phosphate clearing around the colonies was measured. Spores mixed with purified water were used as control. Bacteria-only controls (no fungus) were also performed. Five replicates were performed for each bacterium. Example calcium phytate agar plates are shown in FIG. 10.

On the calcium phytate agar plates, the data showed that, after 2 days, strains 346, 365 and 371 all increased the ability of the fungus to solubilize phytate, based on the clearing zones. At 2 days, the bacteria-only controls showed that none of the bacteria alone were able to solubilize phytate. Penicillium bilaiae alone only started to solubilize phytate after 3 days. The data, after 7 days, are shown in Table 2, row 8, and show that strains 313, 346, 365 and 371 were significantly different from the control in this assay.

### Example 8. Effect of Bacteria on Inorganic Phosphate Solubilization by Fungus

The effect of the bacterial strains on the ability of Penicillium bilaiae to solubilize inorganic phosphate was tested. To test solubilization of inorganic phosphate, Sperber agar plates were used. Penicillium bilaiae spores were mixed with bacterial suspensions and added to plates as described in Example 7. After 7 days incubation of the plates at 26° C., the zone of phosphate clearing around the colonies was measured. Spores mixed with purified water were used as control. Bacteria-only controls (no fungus) were also used. Example Sperber agar plates are shown in FIG. 11.

The data showed that bacterial strains 313 and 346 solubilized amounts of phosphate significantly different than the control (Table 2, row 9). Bacteria-only controls showed that bacteria alone were not able to solubilize either phosphate or phytate in these assays.

A second assay for quantifying the amount of inorganic phosphate solubilized, here in solution, was used. Briefly, Penicillium bilaiae, bacterial strains 313, 371, or both 313 and 371, or combinations of Penicillium bilaiae and the bacterial strains, were added to wells of 96-well plates, in NBRIP medium (10 g glucose, 5 g calcium phosphate, 5 g magnesium chloride hexahydrate, 0.25 g magnesium sulfate heptahydrate, 0.20 g potassium chloride and 0.10 g ammonium sulfate, all per liter of purified water). The 96-well plates were incubated at various temperatures (10, 18, 25, 30 or 35° C.) for various times (up to 5 days). The plates were centrifuged to pellet the cells in the wells. The supernatants were removed and assayed for free phosphate after incubation with malachite green reagent for 30 min. and determination of optical density at 650 nm. Controls were used and background optical density was determined.

The results obtained using these assays showed that bacterial strains 313, 371, or a combination of 313 and 371, were not able to solubilize phosphate. Penicillium bilaiae did solubilize phosphate in absence of the bacteria, but increased the amount of phosphate solubilized, and increased the rate at which phosphate was solubilized when the bacteria were present. Using this assay, phosphate solubilization by Penicillium bilaiae was found to be affected by temperature. Even at 3 days, phosphate solubilized by Penicillium bilaiae alone at 10° C. and 18° C. was not detectable. In the presence of the bacteria, phosphate solubilized by Penicillium bilaiae, at 10° C. was detectable after 2 days and, at 18° was detectable at 0.5 days. At 35° C., phosphate solubilized by Penicillium bilaiae alone was detectable only after 2 days. In combination with the bacteria, phosphate solubilized by Penicillium bilaiae was detected earlier, at 0.5 days. These temperature data indicate that the bacteria decrease the lower temperature and increase the higher temperature at which the fungus may solubilize phosphate, or increase the efficiency with which the fungus may solubilize phosphate at those temperatures.

### Example 9. Effect of Bacteria and Fungus on Plant Growth

To determine whether a composition of one or more of the bacterial strains isolated using the disclosed methods, here strain 313 (DSM 32170) and strain 371 (DSM 32174), and Penicillium bilaiae, when supplied to plants, facilitate plant growth, the study described below was performed. Bacterial strains 313 and 371, and Penicillium bilaiae strains P-201 and P-208 were used. The bacteria and/or Penicillium bilaiae were applied to canola seeds using a commercial seed treater. Bacterial strains 313 and 371 were mixed 1-to-1 and applied to canola seeds at a titer of 1×10⁶ CFU of bacteria per seed. Penicillium bilaiae strains were applied to seeds together, or the P-201 strain was applied alone, at a titer of 5.5×10⁵ CFU of Penicillium bilaiae per seed.

The coated canola seeds were planted in 1-gallon pots that contained watered Fafard® potting media. Three seeds were planted per treatment and were thinned to one plant per pot after emergence. Plants were harvested at approximately 10 weeks after planting. At harvest, pods were collected from each plant, counted and weighed. Plants and pods were then bagged separately, dried in ovens for approximately 1 week at 80° C., and then weighed.

Results are shown in the tables below. In each table, seeds indicated as coated with bacteria were coated with 1×10⁶ CFU per seed of a mixture containing generally equal amounts of strains 313 and 371. Seeds coated with Penicillium bilaiae were coated with either 5.5×10⁵ CFU per seed of strain P-201, or 5.5×10⁵ CFU per seed of a mixture containing generally equal amounts of strains P-201 and P-208.

In each table, statistically-significant differences between numbers (α of 0.05 using Tukey's test) are indicated by different letters in the columns labeled “stats” (i.e., different values within a table with the same letter are not significant at this α).

TABLE 3
Pod numbers from plants grown from coated canola seeds

| | Mean pod number per plant | |
|---|---|---|
| | (values in parentheses were | |
| | normalized to numbers with no | |
| Seed coating | seed coating) | Stats |
| None | 17.083 (1.0) | B |
| Bacteria | 22.917 (1.3) | B |
| P-201 | 21.333 (1.2) | B |
| P-201 + bacteria | 19.917 (1.2) | B |
| P-201 + P-208 | 26.667 (1.6) | B |
| P-201 + P-208 + bacteria | 50.917 (3.0) | A |

TABLE 4
Pod fresh weight from plants grown from coated canola seeds

| | Mean total weight of pods per | |
|---|---|---|
| | plant in grams (values in | |
| | parentheses were normalized to | |
| Seed coating | weights with no seed coating) | Stats |
| None | 4.336 (1.0) | B |
| Bacteria | 5.463 (1.3) | B |
| P-201 | 5.410 (1.2) | B |
| P-201 + bacteria | 4.386 (1.0) | B |
| P-201 + P-208 | 6.106 (1.4) | B |
| P-201 + P-208 + bacteria | 11.298 (2.6) | A |

TABLE 5
Pod dry weight from plants grown from coated canola seeds

| | Mean total dry weight of pods | |
|---|---|---|
| | per plant in grams (values in | |
| | parentheses were normalized to | |
| Seed coating | weights with no seed coating) | Stats |
| None | 0.902 (1.0) | B |
| Bacteria | 1.320 (1.5) | B |
| P-201 | 1.269 (1.4) | B |
| P-201 + bacteria | 1.103 (1.2) | B |
| P-201 + P-208 | 1.350 (1.5) | B |
| P-201 + P-208 + bacteria | 2.322 (2.6) | A |

TABLE 6
Plant dry weight (minus pods) from plants grown from coated canola seeds

| | Mean total dry weight of | |
|---|---|---|
| | individual plants in grams | |
| | (values in parentheses were | |
| | normalized to weights with no | |
| Seed coating | seed coating) | Stats |
| None | 17.825 (1.0) | D |
| Bacteria | 20.498 (1.1) | BCD |
| P-201 | 24.555 (1.4) | AB |
| P-201 + bacteria | 19.375 (1.1) | CD |
| P-201 + P-208 | 22.738 (1.3) | BC |
| P-201 + P-208 + bacteria | 28.945 (1.6) | A |

The data show that the bacteria isolated using the methods disclosed herein, in combination with fungi, can have a synergistic effect on plant growth. That is, the effect of the combination on plant growth, here measured by numbers of pods and biomass of pods from canola plants, and biomass of the canola plants, was greater than the effects of either the bacteria or the fungi alone on those parameters. In other studies, plants like spring wheat and oil seed rape have been shown to have increased biomass with combinations of the isolated bacteria and fungi, over any increases seen with the bacteria alone or fungi alone.

While example compositions, methods, and so on have been illustrated by description, and while the descriptions are in considerable detail, it is not the intention of the applicants to restrict or in any way limit the scope of the application. It is, of course, not possible to describe every conceivable combination of components or methodologies for purposes of describing the compositions, methods, and so on described herein. Additional advantages and modifications will readily appear to those skilled in the art. Therefore, the invention is not limited to the specific details and illustrative examples shown and described. Thus, this application is intended to embrace alterations, modifications, and variations that fall within the scope of the application. Furthermore, the preceding description is not meant to limit the scope of the invention.

To the extent that the term “includes” or “including” is employed in the detailed description or the claims, it is intended to be inclusive in a manner similar to the term “comprising” as that term is interpreted when employed as a transitional word in a claim. Furthermore, to the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

### Example Embodiments of the Invention

1. A method, comprising:

identifying an association of different microbes; and

determining whether a first microbe in the association affects growth of a second microbe in the association.

2. The method of embodiment 1, including screening the association for a secondary characteristic.
3. The method of any one of embodiments 1-2, where the microbes come from an environmental sample.
4. The method of any one of embodiments 1-3, where the microbes come from a soil sample.
5. The method of any one of embodiments 1-4, where the identifying uses a close-to-natural system.
6. The method of any one of embodiments 1-5, where the determining is performed in absence of additional nutrients.
7. The method of one of embodiments 1-6, where the first microbe includes a bacterium and the second microbe includes a fungus.
8. The method of one of embodiments 1-7, where the second microbe includes a non-mycorrhizal fungus.
9. The method of any one of embodiments 2-8, where the secondary characteristic includes increased phosphate solubilization as compared to the first microbe and second microbe alone.
10. The method of any one of embodiments 2-9, where the secondary characteristic includes a capability to facilitate plant growth.
11. The method of any one of embodiments 1-10, where the first microbe in the association facilitates growth of the second microbe.
12. A method, comprising:

isolating a bacterium that associates with a fungus; and

screening for one or both of:
i) capability of the bacterium to affect growth of the fungus; and
ii) capability of the fungus to affect growth of the bacterium.
   13. The method of embodiment 12, including:

testing an association of the bacterium and the fungus for a characteristic that is not growth of the fungus or growth of the bacterium.

14. The method of embodiment 13, where the characteristic that is not growth of the fungus or growth of the bacterium includes an increase in phosphate solubilization as compared to phosphate solubilization by either the fungus or the bacterium alone.
15. The method of any one of embodiments 13-14, where the characteristic that is not growth of the fungus or growth of the bacterium includes capability to facilitate plant growth better than the capability of either the fungus or the bacterium alone to facilitate plant growth.
16. The method of any one of embodiments 12-15, where the fungus is a non-mycorrhizal fungus.
17. The method of any one of embodiments 12-16, where the isolating is performed under close-to-natural conditions.
18. The method of any one of embodiments 12-17, where the screening is performed under conditions where nutrients other than those in agar are not provided to the bacterium or fungus.
19. A method, comprising:

isolating a bacterium that associates with a fungus; and

screening the bacterium for capability to affect growth of the fungus.

20. The method of embodiment 19, where the fungus includes a non-mycorrhizal fungus.
21. The method of any one of embodiments 19-20, where the fungus includes one or more of, Aspergillis, Fusarium, Alternaria, Achrothicium, Arthrobotrys, Penicillium, Cephalosporium, Cladosprium, Curvularia, Cunnighamella, Candida, Chaetomium, Humicola, Helminthosporium, Paecilomyces, Pythium, Phoma, Populospora, Myrothecium, Morteirella, Micromonospora, Oideodendron, Rhizoctonia, Rhizopus, Mucor, Talaromyces, Trichoderma, Torula, Schwanniomyces and Sclerotium.
22. The method of any one of embodiments 19-21, where the fungus includes one or more of, Arthrobotrys oligospora, Aspergillus awamori, Aspergillus niger, Aspergillus tereus, Aspergillus flavus, Aspergillus nidulans, Aspergillus foetidus, Aspergillus wentii, Fusarium oxysporum, Alternaria teneius, Penicillium digitatum, Penicillium lilacinium, Penicillium bilaiae, Penicillium funicolosum, Curvularia lunata, Chaetomium globosum, Humicola inslens, Humicola lanuginosa, Paecilomyces fusisporous, Populospora mytilina, Myrothecium roridum, Rhizoctonia solani, Trichoderma viridae, Torula thermophila, Schwanniomyces occidentalis and Sclerotium rolfsii.
23. The method of any one of embodiments 19-22, where the fungus includes Penicillium bilaiae.
24. The method of any one of embodiments 19-23, where the bacterium that associates with a fungus associates with a hyphae of the fungus.
25. The method of any one of embodiments 19-24, where the fungus is capable of solubilizing phosphate.
26. The method of any one of embodiments 19-25, where screening the bacterium for the capability to affect growth of the fungus is performed under conditions where additional nutrients are not provided to the bacterium or fungus.
27. The method of any one of embodiments 19-26, where screening the bacterium for the capability to affect growth of the fungus includes:

placing the fungus and the bacterium on a support, so that the fungus and the bacterium do not contact one another, and so that the fungus and the bacterium are proximate to one another, so that an effect of the bacterium on growth of the fungus is capable of being detected.

28. The method of embodiment 27, where the support includes water agar medium.
29. The method of any one of embodiments 19-28, including:

testing the bacterium for capability to affect a property of the fungus that is not growth of the fungus.

30. The method of embodiment 29, where the property of the fungus that is not growth of the fungus, includes capability of the fungus to affect a plant.
31. The method of any one of embodiments 29-30, where the property of the fungus that is not growth of the fungus, includes capability of the fungus to facilitate plant growth.
32. The method of embodiment 31, where a fungus having the capability to facilitate plant growth is capable of one or both of, i) providing nutrients to the plant, and ii) at least partially preventing effects of a plant pathogen on the plant.
33. The method of any one of embodiments 31-32, where a fungus having the capability to facilitate plant growth is capable of solubilizing phosphate.
34. The method of embodiment 29, where the property of the fungus that is not growth of the fungus, includes capability of the fungus to solubilize phosphate.
35. The method of any one of embodiments 19-34, including:

testing the bacterium for capability, in combination with the fungus, to increase phosphate solubilization, as compared to phosphate solubilization by the fungus alone.

36. The method of any one of embodiments 19-35, including:

testing the bacterium for capability, in combination with the fungus, to increase facilitation of plant growth, as compared to facilitation of plant growth by the fungus alone.

37. The method of any one of embodiments 19-36, where the isolating simulates a close-to-natural system.
38. The method of any one of embodiments 19-37, where isolating a bacterium that associates with a fungus includes:

establishing the fungus on a support;

contacting the support with a soil microcosm; and

obtaining a bacterium attached to the fungus.

39. The method of embodiment 38, where the support includes a glass or polycarbonate support.
40. The method of any one of embodiments 38-39, where obtaining the bacterium associated with the fungus includes:

washing the support so that bacteria not attached to the fungus are removed; and

culturing the bacterium attached to the fungus.

41. The method of one of embodiments 19-40, where a bacterium that is capable of affecting growth of the fungus is capable of stimulating growth of the fungus.
42. The method of embodiment 41, where stimulating growth of the fungus includes enhancing germination of spores of the fungus.
43. The method of any one of embodiments 19-42, where the bacterium includes one or more of strains 313 (DSM 32170), 346 (DSM 32171), 351 (DSM 32172), 365 (DSM 32173) and 371 (DSM 32174).
44. The method of any one of embodiments 19-40, where a bacterium that is capable of affecting growth of the fungus is capable of impeding growth of the fungus.
45. A bacterium obtained by the method of any one of embodiments 1-44.
46. A method, comprising:

enriching a population of microbes in an environmental sample for bacteria that colonize non-mycorrhizal fungi; and

examining the bacteria that colonize the non-mycorrhizal fungi for an ability to promote growth of the fungi.

47. The method of embodiment 46, including:

isolating from the bacteria that possess the ability to promote growth of the fungi, bacteria that, with the fungi, are better able to solubilize phosphate than either the bacteria or fungi alone.

48. The method of any one of embodiments 46-47, where the enriching uses a close-to-natural soil system.
49. The method of any one of embodiments 46-48, where the bacteria are not capable of solubilizing phosphate alone.
50. The method of one of embodiments 46-48, where the bacteria are capable of solubilizing phosphate alone.
51. A method, comprising:

isolating a bacterium that forms an association with, and enhances growth of, a non-mycorrhizal fungus, the association having a secondary characteristic; and

marketing the bacterium and the non-mycorrhizal fungus, together or separately, for use as a combination.

52. The method of embodiment 51, where the bacterium is isolated from a soil sample using a close-to-natural system.
53. The method of any one of embodiments 51-52, where the bacterium enhances growth of the non-mycorrhizal fungus when nutrients other than those provided by agar are not present.
54. The method of any one of embodiments 51-53, where the bacterium enhances growth of the non-mycorrhizal fungus when nutrients other than those provided by agar at a concentration of about 1.5% weight/volume are not present.
55. The method of any one of embodiments 51-54, where the secondary characteristic includes increased phosphate solubilization as compared to the bacterium and the non-mycorrhizal fungus alone.
56. The method of any one of embodiments 51-55, where marketing includes one or more of promoting, advertising, storing, offering to sell, selling and shipping, the bacterium and non-mycorrhizal fungus.
57. The method of any one of embodiments 51-56, where the secondary characteristic includes a capability to facilitate plant growth.
58. The method of any one of embodiments 51-57, where the use includes supplying to a plant.
59. A method, comprising:

isolating from an environmental sample, a bacterium that associates with a hyphae of a non-mycorrhizal fungus and affects growth of the fungus; and

combining the bacterium and the fungus.

60. The method of embodiment 59, where the environmental sample includes a soil sample.
61. The method of any one of embodiments 59-60, where the isolating is performed in a close-to-natural soil system.
62. The method of any one of embodiments 59-61, where the bacterium that affects growth of the fungus, facilitates growth of the fungus.
63. The method of embodiment 62, where the bacterium facilitates growth of the fungus when nutrients not supplied by agar at a concentration of up to 1.5% are not present.
64. The method of one of embodiments 59-63, where the combining forms a combination.
65. The method of embodiment 64, where the combination includes a kit.
66. The method of embodiment 65, where the kit includes a first container housing the bacterium and a second container housing the Penicillium bilaiae.
67. The method of any one of embodiments 64-66, where the combination is supplied to a plant.
68. The method of any one of embodiments 64-67, where the combination is applied to one or both of a seed, and a furrow in which a seed or seedling is planted.
69. The method of any one of embodiments 64-68, where the combination is capable of expressing at least one secondary characteristic.
70. The method of any one of embodiments 59-69, where the bacterium includes one or more of strains 313 (DSM 32170), 346 (DSM 32171), 351 (DSM 32172), 365 (DSM 32173) and 371 (DSM 32174).
71. A method, comprising:

obtaining a bacterium that binds to and enhances growth of a Penicillium bilaiae, the bacterium capable of increasing the capability of the Penicillium bilaiae to solubilize phosphate; and

combining the bacterium with the Penicillium bilaiae.

72. The method of embodiment 71, including marketing a combination of the bacterium and the Penicillium bilaiae.
73. The method of any one of embodiments 71-72, where obtaining the bacterium that binds to a Penicillium bilaiae uses a close-to-natural soil-based system.
74. The method of any one of embodiments 71-73, where obtaining the bacterium that enhances growth of a Penicillium bilaiae uses a system that contains no nutrients additional to those present in a medium containing up to 1.5% agar.
75. The method of any one of embodiments 72-74, where the combination of the bacterium and the Penicillium bilaiae is marketed for providing usable phosphate to a plant.
76. The method of any one of embodiments 72-75, where the combination of the bacterium and the Penicillium bilaiae is in the form of a kit.
77. The method of embodiment 76, where the kit includes a first container housing the bacterium and a second container housing the Penicillium bilaiae.
78. The method of any one of embodiments 71-77, where the bacterium includes one or more of strains 313 (DSM 32170), 346 (DSM 32171), 351 (DSM 32172), 365 (DSM 32173) and 371 (DSM 32174).
79. A method, comprising:

combining with a Penicillium, one or more bacteria that associate with, stimulate growth of, and increase the capability of the Penicillium to solubilize phosphate; and

offering the one or more bacteria and the Penicillium for sale.

80. The method of embodiment 79, where the Penicillium is capable of solubilizing phosphate.
81. The method of any one of embodiments 79-80, where one or more of the bacteria are obtained by the method of any one of embodiments 1-80.
82. The method of any one of embodiments 79-81, where the one or more bacteria includes strains 313 (DSM 32170), 346 (DSM 32171), 351 (DSM 32172), 365 (DSM 32173) and 371 (DSM 32174).
83. A composition, comprising a bacterium obtained by the method of any one of embodiments 1-82.
84. The composition of embodiment 83, including at least one excipient.
85. The composition of any one of embodiments 83-84, including at least one synthetic excipient.
86. The composition of any one of embodiments 83-85, where the bacterium includes one or more isolated bacterial strains 313 (DSM 32170), 346 (DSM 32171), 351 (DSM 32172), 365 (DSM 32173) and 371 (DSM 32174).
87. The composition of any one of embodiments 83-86, where the composition includes a water-soluble solid or a liquid having a concentration of the bacterial strains, as measured by colony-forming units (CFU), of at least one of 1×10⁹, 5×10⁹, 1×10¹⁰, 5×10¹⁰ or 1×10¹¹ per gram of the water-soluble solid or per milliliter of the liquid.
88. The composition of any one of embodiments 83-87, where at least one of 50%, 60%, 70%, 80%, 90%, 95% or 99% of the bacterial strains in the composition are in the form of spores.
89. The composition of any one of embodiments 83-88, where the composition includes one or more components from the medium in which the bacterial strains were propagated.
90. The composition of any one of embodiments 83-89, where the composition includes at least one anti-fungal agent.
91. The composition of any one of embodiments 83-90, where the composition includes at least one spore anti-germinant.
92. The composition of any one of embodiments 83-91, including one or more Penicillium bilaiae strains that are capable of solubilizing phosphate.
93. The composition of embodiment 92, where the Penicillium bilaiae has a concentration, as measured by colony-forming units (CFU), of at least one of 5×10⁷, 1×10⁸, 5×10⁸, 1×10⁹ or 5×10⁹ per gram of solid or per milliliter of liquid.
94. The composition of any one of embodiments 92-93, where at least one of 50%, 60%, 70%, 80%, 90%, 95% or 99% of the Penicillium bilaiae in the composition is in the form of spores.
95. A method, comprising:

supplying a composition of any one of embodiments 83-94 to a plant.

96. The method of embodiment 95, where supplying to a plant includes applying the composition to one or both of a seed, or a furrow in which a seed or a seedling is planted.
97. The method of any one of embodiments 95-96, where one or more biostimulants, nutrients, pesticides or plant signal molecules are applied to the seed or the furrow.
98. The method of embodiment 97, where pesticides include one or more acaricides, fungicides, gastropodicides, herbicides, insecticides, nematicides, rodenticides and virucides.
99. The method of any one of embodiments 95-99, including planting the seed.
100. The method of embodiment 99, including growing the seed.
101. A bacterium that cannot solubilize phosphate alone but, in association with a non-mycorrhizal fungus that can solubilize phosphate alone, the bacterium can increase the amount of phosphate solubilized and/or the rate at which phosphate is solubilized, as compared to the fungus alone.
102. The bacterium of embodiment 101, where the amount of phosphate solubilized and/or the rate at which phosphate is solubilized by the association of bacterium and fungus is at least one of 5%, 10%, 20%, 30%, 40%, 50%, 75%, 100%, 200%, 300%, 400% or 500% greater than the amount and/or rate of phosphate solubilized by the fungus alone.
103. The bacterium of embodiments 101-102 that includes at least one of bacterial strains 313 (DSM 32170), 346 (DSM 32171), 351 (DSM 32172), 365 (DSM 32173) and 371 (DSM 32174).
104. A combination of the bacterium and the fungus of embodiments 101-103.
105. A combination of the bacterium and the fungus of embodiments 101-103, where the fungus is a Penicillium.
106. A combination of the bacterium and the fungus of embodiments 101-103, where the fungus is a Penicillium bilaiae.
107. A method, comprising:

enriching a population of microbes in a soil sample for bacteria that associate with a non-mycorrhizal fungus that is added to the soil sample;

testing the bacteria that associate with the fungus, for bacteria that affect growth of the fungus; and

placing the bacteria that affect growth of the fungus together with the non-mycorrhizal fungus to form a mixture, and screening the mixture for a characteristic not present in either the bacteria or non-mycorrhizal fungus alone, or for improvement in a characteristic present in either the bacteria or non-mycorrhizal fungus alone.

108. The method of embodiment 107, where the bacteria that affect growth of the fungus, stimulate growth of the fungus.
109. The method of any of embodiments 107-108, where the testing uses a microbial medium that does not contain nutrients in addition to nutrients provided by bacteriological-grade agar at a concentration between about 0.5-1.5%.
110. The method of any of embodiments 107-109, where a characteristic present in the mixture and not present in either the bacteria or non-mycorrhizal fungus alone, or characteristic present in one or both of the bacteria and the non-mycorrhizal fungus but improved in the mixture, includes: an activity that provides plant-usable nutrients; an activity that inhibits and/or kills a fungus, nematode, bacterium, insect or weed; or an activity that improves stability or viability of a microbe.
111. The method of any of any of embodiments 107-110, where the non-mycorrhizal fungus is capable of solubilizing phosphate alone.
112. A method, comprising:

enriching a population of microbes in a soil sample for bacteria that associate with a hyphae of a Penicillium bilaiae that is added to the soil sample;

testing the bacteria that associate with the hyphae, for bacteria that stimulate growth of the Penicillium bilaiae; and

screening the bacteria that stimulate growth of the Penicillium bilaiae for the capability to increase an amount of phosphate solubilized by the Penicillium bilaiae.

113. The method of embodiment 112, where the testing includes mixing the bacteria and the Penicillium bilaiae to form a mixture and comparing the growth of the Penicillium bilaiae in the mixture with growth of Penicillium bilaiae alone.
114. The method of embodiment 112, where the testing includes placing the bacteria and the Penicillium bilaiae on a support so they do not initially contact one another and, after a period of time, determining whether the bacteria and the Penicillium bilaiae contact each other on the support.
115. The method of one of embodiments 112-114, where the testing uses a microbial medium that does not contain nutrients in addition to nutrients supplied by bacteriological-grade agar at a concentration between about 0.5-1.5%.
116. The method of one of embodiments 112-115, including:

offering for sale, separately or together, the bacteria that both stimulate growth of the Penicillium bilaiae and increase the amount of phosphate solubilized by the Penicillium bilaiae, and the Penicillium bilaiae, for use as a combination.

117. The method of claim 116, including:

supplying the combination to a plant.

118. A method, comprising:

contacting a phosphate-solubilizing Penicillium bilaiae, that has been established on a solid support, with an environmental soil sample under close-to-natural conditions, and culturing bacteria from the soil sample that bind to the fungus on the solid support;

isolating from the bacteria that bind to the Penicillium bilaiae, bacteria that when placed in proximity to, or mixed with, the Penicillium bilaiae, stimulate growth of the Penicillium bilaiae in the presence of no more nutrients than are provided by bacteriological-grade agar at a concentration between about 0.5-1.5%;

screening the bacteria that stimulate growth of the Penicillium bilaiae for bacteria with a capability to increase an amount of phosphate solubilized by the Penicillium bilaiae;

combining the bacteria that increase the amount of phosphate solubilized by the Penicillium bilaiae, with the Penicillium bilaiae to form a combination, and supplying the combination of the bacteria and the Penicillium bilaiae to a plant.

119. The method of one of embodiments 107-118, where the bacteria include at least one of bacterial strains 313 (DSM 32170), 346 (DSM 32171), 351 (DSM 32172), 365 (DSM 32173) and 371 (DSM 32174).
120. A composition, comprising a bacterium obtained by the method of one of embodiments 107-118, and at least one excipient.
121. The composition of embodiment 120, where the bacterium includes at least one of bacterial strains 313 (DSM 32170), 346 (DSM 32171), 351 (DSM 32172), 365 (DSM 32173) and 371 (DSM 32174).
122. The composition of one of embodiments 120-121, including a phosphate-solubilizing Penicillium bilaiae.
123. The composition of one of embodiments 120-122, where the composition is in the form of a liquid, gel, slurry or solid.
124. The composition of embodiment 123, where the solid includes a wettable powder, a dry powder or granules.
125. The composition of one of embodiments 120-124, where the composition is packaged as a kit.
126. The composition of one of embodiments 120-125, where the composition is supplied to a plant by applying the composition to a seed, or to a furrow in which a seed or seedling is planted.

### Deposit of Biological Material

The following biological material has been deposited under the terms of the Budapest Treaty with the Leibniz Institute DSMZ-German Collection of Microorganisms and Cell Cultures, Inhoffenstraße 7B, 38124 Braunschweig, Germany on Oct. 8, 2015 by The University of Copenhagen, and is identified as follows:

Strain 313—DSM 32170; Strain 346—DSM 32171; Strain 351—DSM 32172; Strain 365—DSM 32173; and Strain 371—32174.

The following biological material has been deposited under the terms of the Budapest Treaty with the Agricultural Research Service Patent Culture Collection (NRRL), Northern Regional Research Center, 1815 N. University Street, Peoria, Ill., USA in August 2008, and is identified as follows:

Penicillium bilaiae strain P-201—NRRL 50169 (deposited Aug. 28, 2008); and Penicillium bilaiae strain P-208—NRRL 50162 (deposited Aug. 11, 2008).

1. A method, comprising:
enriching a population of microbes in a soil sample for bacteria that associate with a non-mycorrhizal fungus that is added to the soil sample;
testing the bacteria that associate with the fungus, for bacteria that affect growth of the fungus; and
placing the bacteria that affect growth of the fungus together with the non-mycorrhizal fungus to form a mixture, and screening the mixture for a characteristic not present in either the bacteria or non-mycorrhizal fungus alone, or for improvement in a characteristic present in either the bacteria or non-mycorrhizal fungus alone.

2. The method of claim 1, where the bacteria that affect growth of the fungus, stimulate growth of the fungus.

3-5. (canceled)

6. A method, comprising:
enriching a population of microbes in a soil sample for bacteria that associate with a hyphae of a Penicillium bilaiae that is added to the soil sample;
testing the bacteria that associate with the hyphae, for bacteria that stimulate growth of the Penicillium bilaiae; and
screening the bacteria that stimulate growth of the Penicillium bilaiae for the capability to increase an amount of phosphate solubilized by the Penicillium bilaiae.

7. (canceled)

8. The method of claim 6, where the testing includes placing the bacteria and the Penicillium bilaiae on a support so they do not initially contact one another and, after a period of time, determining whether the bacteria and the Penicillium bilaiae contact each other on the support.

9. The method of claim 8, where the testing uses a microbial medium that does not contain nutrients in addition to nutrients supplied by bacteriological-grade agar at a concentration between about 0.5-1.5%.

10. The method of claim 6, including:
combining the bacteria that both stimulate growth of the Penicillium bilaiae and increase the amount of phosphate solubilized by the Penicillium bilaiae, and the Penicillium bilaiae into a combination;
and supplying the combination to a plant.

11. (canceled)

12. The method of claim 6, where the bacteria that both stimulate growth of the Penicillium bilaiae and increase the amount of phosphate solubilized by the Penicillium bilaiae include at least one of bacterial strains 313 (DSM 32170), 346 (DSM 32171), 351 (DSM 32172), 365 (DSM 32173) and 371 (DSM 32174).

13. A composition, comprising a bacterium obtained by the method of claim 1, and at least one excipient.

14-15. (canceled)

16. The composition of claim 13, including the non-mycorrhizal fungus which is a phosphate-solubilizing Penicillium bilaiae.

17. The composition of claim 16, where the composition is capable of solubilizing phosphate at a higher rate than the Penicillium bilaiae alone at temperatures at least between 10° C. and 35° C.

18-19. (canceled)

20. The composition of claim 17, where the composition is supplied to a plant by applying the composition to a seed, or to a furrow in which a seed or seedling is planted.

21. (canceled)

22. The composition of claim 17, where the composition is supplied to a canola plant and increases at least one of pod count, pod fresh weight, pod dry weight, or plant dry weight, synergistically, as compared to supplying the bacterium, bacterial strain, or Penicillium bilaiae alone to the canola plant.

23. (canceled)

24. The composition of claim 16, where the phosphate-solubilizing Penicillium bilaiae includes strain P-201.

25. The composition of claim 16, where the phosphate-solubilizing Penicillium bilaiae includes both strain P-201 and P-208.

26. A method of increasing plant yield, comprising:
combining a bacterium that both stimulates growth of a Penicillium bilaiae and increases the amount of phosphate solubilized by the Penicillium bilaiae, with Penicillium bilaiae to form a combination; and
supplying the combination to a plant.
